(19) United States
(12) Patent Application Publication
(10) Pub. No.: US 2022/0208361 A1
(43) Pub. Date: Jun. 30, 2022

(54) CARE SUPPORT SYSTEM, CARE SUPPORT INFORMATION REGISTRATION SYSTEM, CARE SUPPORT METHOD, AND CARE SUPPORT INFORMATION REGISTRATION METHOD

(71) Applicant: Shelpa Co. Ltd., Kanagawa-ku, Yokohama-shi, Kanagawa (JP)

(72) Inventor: Youichi ISHIKAWA, Kanagawa-ku, Yokohama-shi, Kanagawa (JP)

(21) Appl. No.: 17/612,080

(22) PCT Filed: May 14, 2020
(86) PCT No.: PCT/JP2020/019187

§ 371 Date: Nov. 17, 2021

(30) Foreign Application Priority Data
May 20, 2019 (JP) 2019-094484
Jan. 20, 2020 (JP) 2020-007130

(51) Int. Cl. G16H 40/20; G16H 20/00; G16H 15/00

(52) U.S. Cl. CPC G16H 40/20; G16H 15/00; G16H 20/00

(57) ABSTRACT

A care support system is provided which allows even a care practitioner with little experience to create a care plan, or implement care, for fulfilling a desire of a care target person.

A care support apparatus 100 inputs a desire of a care target person, displays candidates for a goal for fulfilling the inputted desire on the basis of a goal information table 420, inputs any of the displayed goals, displays one or more issues in achieving the inputted goal on the basis of an issue information table 440, inputs any of the displayed issues, and registers, in a storage device 42, care management information including the name of the care target person, and the inputted desire, goal, and issue. The name, the desire, the goal, and the issue are displayed per care target person on the basis of the care management information of the storage device 42.

### TECHNICAL FIELD

The present invention relates to a system and method for supporting a care practitioner in implementing care for a care target person, and particularly relates to a care support system, care support information registration system, care support method, and care support information registration method that allows even a care practitioner with little experience to create a care plan, or implement care, for fulfilling a desire of a care target person.

### BACKGROUND ART

For example, a technology described in Patent Literature 1 is conventionally known as a technology for supporting a care practitioner such as a care manager in implementing care for a care target person such as an elderly person.

The technology described in Patent Literature 1 is configured in such a manner as to perform processes in the order of registering in advance a combination master, a problem identification condition master, and a problem identification master, creating a face sheet and assessment table for an individual targeted for care, retrieving from the assessment table an assessment value corresponding to a determination period and a determination condition on the problem identification condition master, retrieving from the combination master a problem code of a problem condition corresponding to the assessment value, identifying a problem area, a problem point, a weight, a care goal, and a problem, which correspond to the problem code, and the problem code on the basis of the contents of the problem identification master, and creating a table sequentially. Consequently, it is possible to create an assessment table, identify a problem area, a problem, and a case, and create a care plan whenever necessary.

### CITATION LIST

### Patent Literature

PATENT LITERATURE 1: JP-A-10-334147

### SUMMARY OF INVENTION

### Problems to be Solved by Invention

However, in the technology described in Patent Literature 1, a care plan is created on the basis of a problem and a goal, which are set by a care practitioner. Accordingly, the result of implementation of care may not be in consonance with a desire of a care target person. For example, if a care target person has a desire to not live at a facility but go back home, a care plan should be ideally created in such a manner as to fulfill the desire. However, if a uniform care plan is created, focusing only on the physical functioning of the care target person and problems thereof, as described in [0016] to [0019] in Patent Literature 1, the desire is far from being fulfilled, which leads to a reduction in the degree of satisfaction of the care target person and his/her family.

On the other hand, that being said, focusing only on physical functioning and problems thereof as described above is not sufficient to create a care plan for fulfilling a desire of a care target person. A care practitioner requires rich experience. Accordingly, it is difficult for a care practitioner with little experience to create a care plan.

Hence, the present invention has been made, focusing on the unsolved issues of such a known technology, and an object thereof is to provide a care support system, care support information registration system, care support method, and care support information registration method that allows even a care practitioner with little experience to create a care plan, or implement care, for fulfilling a desire of a care target person.

### Solutions to Problems

### [Aspect A1]

Aiming at achieving the above-mentioned object, the care support system of aspect A1 is a care support system that supports a care practitioner in implementing care for a care target person, the care support system comprising: a desire input means configured to input a desire of the care target person or his/her family; a goal presenting means configured to present one or more goals for fulfilling the desire inputted by the desire input means on the basis of goal information, which indicates one or more goals for fulfilling the desire of the care target person or his/her family, of a goal information storage means where the goal information is stored, associated with the desire; a goal input means configured to input any of the goals presented by the goal presenting means; an issue presenting means configured to present one or more issues in achieving the goal inputted by the goal input means on the basis of issue information, which indicates one or more issues in achieving the goal related to the goal information, of an issue information storage means where the issue information is stored, associated with the goal; an issue input means configured to input any of the issues presented by the issue presenting means; an identification information input means configured to input identification information for identifying the care target person; a care management information registration means configured to register, in a care management information storage means, care management information including the identification information inputted by the identification information input means, the desire inputted by the desire input means, the goal inputted by the goal input means, and the issue inputted by the issue input means; and a care management information presenting means configured to present the identification information, the desire, the goal, and the issue per care target person on the basis of the care management information of the care management information storage means.

The goal information storage means stores the goal information with every means and at every timing. The goal information storage means may store the goal information in advance, or may be configured in such a manner as to store the goal information in response to, for example, input from the outside during operation of the system without storing the goal information in advance. The same shall apply to other storage means.

Moreover, storing the goal information while associating the goal information with the desire includes, for example, (1) storing the goal information while associating the goal information directly with the desire by, for example, registering the goal information and the desire in the same record, and (2) storing the goal information via one or more pieces of information in between by, for example, providing a table where the goal information and intermediate information are associated and registered, and a table where the desire and the intermediate information are associated and registered. In other words, an aspect where the goal information can be traced from the desire may accept any data structure. In terms of the concept that information is associated and stored, the same shall apply below.

Moreover, the system may be realized as a single apparatus, terminal, or other devices, or as a network system that connects a plurality of apparatuses, terminals, or other devices in such a manner as to be able to communicate with each other. In the latter case, each constituent element may belong to any of, for example, the plurality of devices as long as being connected to be able to communicate. The same shall apply below in a care support system according to aspects B1, B14, and B15, and a care support information registration system according to aspects B16 to B20.

### [Aspect A2]

Furthermore, the care support system of aspect A2 is the care support system according to aspect A1, further comprising: an achievement degree input means configured to input the degree of achievement of the goal inputted by the goal input means; and a fulfillment degree input means configured to input the degree of fulfillment of the desire inputted by the desire input means, wherein the care management information registration means registers, in the care management information storage means, the care management information including the identification information, the desire inputted by the desire input means, the goal inputted by the goal input means, the issue inputted by the issue input means, the degree of achievement inputted by the achievement degree input means, and the degree of fulfillment inputted by the fulfillment degree input means, and the care management information presenting means presents the identification information, the desire, the goal, the issue, the degree of achievement, and the degree of fulfillment per care target person on the basis of the care management information of the care management information storage means.

The degree of achievement indicates the extent to which a goal has been achieved, and includes, for example, one indicating whether or not a goal has been achieved (success or failure in achievement), one indicating the ratio of the degree of achievement of a goal to complete achievement of the goal (for example, in percentage), one indicating a value that increases as the achievement nears, and one indicating a value that decreases as the achievement nears. The degree of achievement can be expressed by, for example, a numeric value, a character, a symbol, graphics, an image, and other identifiers. The same shall apply below in terms of the degree of achievement.

Moreover, the degree of fulfillment indicates the extent to which a desire has come true, and includes, for example, one indicating whether or not a desire has come true (success or failure in fulfillment), one indicating the ratio of the degree of fulfillment of a desire to complete fulfillment of the desire (for example, in percentage), one indicating a value that increases as the fulfillment nears, and one indicating a value that decreases as the fulfillment nears. The degree of fulfillment can be expressed by, for example, a numeric value, a character, a symbol, graphics, an image, and other identifiers. The same shall apply below in terms of the degree of fulfillment.

### [Aspect A3]

Furthermore, the care support system of aspect A3 is the care support system according to aspect A2, further comprising: a target achievement date input means configured to input a target achievement date scheduled to achieve the goal inputted by the goal input means; an update input means configured to input an update of a previous input; an actual fulfillment date input means configured to input a fulfillment date when the desire inputted by the desire input means is actually fulfilled; and an actual achievement date input means configured to input an achievement date when the goal inputted by the goal input means is actually achieved, wherein the care management information registration means registers, in the care management information storage means, the care management information including the identification information, the desire inputted by the desire input means, the goal inputted by the goal input means, the issue inputted by the issue input means, the degree of achievement inputted by the achievement degree input means, the degree of fulfillment inputted by the fulfillment degree input means, the target achievement date inputted by the target achievement date input means, the update inputted by the update input means, the actual fulfillment date inputted by the actual fulfillment date input means, and the actual achievement date inputted by the actual achievement date input means, and the care management information presenting means presents the identification information, the desire, the goal, the issue, the degree of achievement, the degree of fulfillment, the target achievement date, the update, the actual fulfillment date, and the actual achievement date per care target person on the basis of the care management information of the care management information storage means.

### [Aspect A4]

Furthermore, the care support system of aspect A4 is the care support system according to either aspect A2 or aspect A3, wherein the goal information storage means stores goal information indicating one or more goals for fulfilling the desire of the care target person or his/her family, and the degrees of difficulty in achieving the one or more goals, associating the goal information with the desire, and upon the degree of achievement inputted by the achievement degree input means or the degree of fulfillment inputted by the fulfillment degree input means, both of which are related to the care target person, being equal to or less than a predetermined degree, the goal presenting means presents a goal whose degree of difficulty in achievement corresponding to the goal is equal to or less than a predetermined degree among one or more goals for fulfilling the desire of the care target person inputted by the desire input means, on the basis of the goal information of the goal information storage means.

### [Aspect A5]

Furthermore, the care support system of aspect A5 is the care support system according to either aspect A2 or aspect A3, further comprising an action item presenting means configured to present an action item in accordance with the degree of achievement inputted by the achievement degree input means, or the degree of fulfillment inputted by the fulfillment degree input means, on the basis of action item information, which indicates an action item necessary to achieve the goal, of an action item information storage means where the action item information is stored, associated with the degree of achievement or the degree of fulfillment.

### [Aspect A6]

Furthermore, the care support system of aspect A6 is the care support system according to any one of aspects A1 to A3, further comprising a recommended goal presenting means configured to present a goal recommended to the care target person among one or more goals for fulfilling the desire of the care target person inputted by the desire input means, on the basis of care track record information, which indicates a care track record of the care target person, of a care track record information storage means where the care track record information is stored, and the goal information of the goal information storage means.

### [Aspect A7]

Furthermore, the care support system of aspect A7 is the care support system according to any one of aspects A1 to A3, further comprising: an attribute information acquisition means configured to acquire attribute information indicating an attribute of a first care target person; and a recommended goal presenting means configured to acquire care track record information indicating a care track record of the care target person, the care track record information corresponding to an attribute having the same or a predetermined relationship with the first care target person, from a care track record information storage means where the care track record information is stored, associated with an attribute of the care target person, on the basis of the attribute information acquired by the attribute information acquisition means, and to present a goal recommended to the first care target person among one or more goals for fulfilling a desire of the first care target person inputted by the desire input means, on the basis of the acquired care track record information and the goal information of the goal information storage means.

### [Aspect A8]

Furthermore, the care support system of aspect A8 is the care support system according to any one of aspects A1 to A3, further comprising: an attribute information acquisition means configured to acquire attribute information indicating an attribute of a first care practitioner; and a recommended goal presenting means configured to acquire care track record information indicating a track record of care implemented by the care practitioner for the care target person, the care track record information corresponding to an attribute having the same or a predetermined relationship with the first care practitioner, from a care track record information storage means where the care track record information is stored, associated with an attribute of the care practitioner, on the basis of the attribute information acquired by the attribute information acquisition means, and to present a goal recommended to the first care practitioner among one or more goals for fulfilling a desire of a care target person for whom care is implemented by the first care practitioner, the desire being inputted by the desire input means, on the basis of the acquired care track record information and the goal information of the goal information storage means.

### [Aspect A9]

Furthermore, the care support system of aspect A9 is the care support system according to either aspect A2 or aspect A3, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or less than, or, being equal to or greater than, a predetermined value, present a goal recommended to the care target person among one or more goals for fulfilling the desire of the care target person inputted by the desire input means, on the basis of the goal information of the goal information storage means.

### [Aspect A10]

Furthermore, the care support system of aspect A10 is the care support system according to aspect A9, wherein the goal information storage means stores goal information indicating one or more goals for fulfilling the desire of the care target person or his/her family, and the degrees of difficulty in achieving the one or more goals, associating the goal information with the desire, and upon the difference or the rank being equal to or greater than the predetermined value, the recommended goal presenting means presents a goal having a different degree of difficulty in achievement from a previous input.

### [Aspect A11]

Furthermore, the care support system of aspect A11 is the care support system according to aspect A3 including a goal achievement determination means configured to determine whether or not the goal inputted by the goal input means can be achieved on the basis of care management information of the care management information storage means related to a goal, an issue, and a target achievement date, which were inputted in the past, and the goal inputted by the goal input means, the issue inputted by the issue input means, and the target achievement date inputted by the target achievement date input means.

### [Aspect A12]

Meanwhile, aiming at achieving the above-mentioned object, the care support method of aspect A12 is a care support method for supporting a care practitioner in implementing care for a care target person, the care support method comprising: a desire input step of causing a desire input means to input a desire of the care target person or his/her family; a goal presenting step of causing a goal presenting means to present one or more goals for fulfilling the desire inputted in the desire input step, on the basis of goal information, which indicates one or more goals for fulfilling the desire of the care target person or his/her family, of a goal information storage means where the goal information is stored, associated with the desire; a goal input step of causing a goal input means to input any of the goals presented in the goal presenting step; an issue presenting step of causing an issue presenting means to present one or more issues in achieving the goal inputted in the goal input step, on the basis of issue information, which indicates one or more issues in achieving the goal related to the goal information, of an issue information storage means where the issue information is stored, associated with the goal; an issue input step of causing an issue input means to input any of the issues presented in the issue presenting step; a care management information registration step of causing a care management information registration means to register, in a care management information storage means, care management information including the identification information for identifying the care target person, the desire inputted in the desire input step, the goal inputted in the goal input step, and the issue inputted in the issue input step; and a care management information presenting step of causing a care management information presenting means to present the identification information, the desire, the goal, and the issue per care target person on the basis of the care management information of the care management information storage means.

### [Aspect B1]

Meanwhile, aiming at achieving the above-mentioned object, the care support system of aspect B1 is a care support system that supports a care practitioner in implementing care for a care target person, the care support system comprising: a desire input means configured to input a desire of the care target person or his/her family; a goal presenting means configured to present one or more goals for fulfilling the desire inputted by the desire input means on the basis of goal information, which indicates one or more goals for fulfilling the desire of the care target person or his/her family, of a goal information storage means where the goal information is stored, associated with the desire; a goal input means configured to input any of the goals presented by the goal presenting means; an action item presenting means configured to present one or more action items necessary to achieve the goal inputted by the goal input means on the basis of action item information, which indicates one or more action items necessary to achieve the goal related to the goal information, of an action item information storage means where the action item information is stored, associated with the goal; an action item input means configured to input any of the action items presented by the action item presenting means; an identification information input means configured to input identification information for identifying the care target person; a care management information registration means configured to register, in a care management information storage means, care management information including the identification information inputted by the identification information input means, the desire inputted by the desire input means, the goal inputted by the goal input means, and the action item inputted by the action item input means; and a care management information presenting means configured to present the identification information, the desire, the goal, and the action item per care target person on the basis of the care management information of the care management information storage means.

The action item is an item necessary to achieve the goal, and includes an item necessary to solve an issue in achieving the goal. Hence, it is assumed that the action item information is stored, associated with the goal or issue. Specifically, the assumptions include, for example, (1) the action item information and the goal are stored, associated directly with each other by, for example, being registered in the same record, (2) the action item information and the issue are stored, associated directly with each other by, for example, being registered in the same record, (3) the action item information is stored via one or more pieces of information in between by, for example, providing a table where the action item information and intermediate information are associated and registered, and a table where the goal and the intermediate information (such as issue information indicating the issue) are associated and registered, and (4) the action item information is stored via one or more pieces of information in between by, for example, providing a table where the action item information and intermediate information are associated and registered, and a table where the issue and the intermediate information are associated and registered. In other words, an aspect where the action item information can be traced from the goal or issue may accept any data structure. The same shall apply below in terms of the action item.

### [Aspect B2]

Furthermore, the care support system of aspect B2 is the care support system according to aspect B1, further comprising: an issue presenting means configured to present one or more issues in achieving the goal inputted by the goal input means on the basis of issue information, which indicates one or more issues in achieving the goal related to the goal information, of an issue information storage means where the issue information is stored, associated with the goal; and an issue input means configured to input any of the issues presented by the issue presenting means, wherein the care management information registration means registers, in the care management information storage means, the care management information including the identification information inputted by the identification information input means, the desire inputted by the desire input means, the goal inputted by the goal input means, the issue inputted by the issue input means, and the action item inputted by the action item input means, and the care management information presenting means presents the identification information, the desire, the goal, the issue, and the action item per care target person on the basis of the care management information of the care management information storage means.

### [Aspect B3]

Furthermore, the care support system of aspect B3 is the care support system according to either aspect B1 or aspect B2, further comprising: an achievement degree input means configured to input the degree of achievement of the goal inputted by the goal input means; and a fulfillment degree input means configured to input the degree of fulfillment of the desire inputted by the desire input means, wherein the care management information registration means registers, in the care management information storage means, the care management information including the identification information inputted by the identification information input means, the desire inputted by the desire input means, the goal inputted by the goal input means, the action item inputted by the action item input means, the degree of achievement inputted by the achievement degree input means, and the degree of fulfillment inputted by the fulfillment degree input means, and the care management information presenting means presents the identification information, the desire, the goal, the action item, the degree of achievement, and the degree of fulfillment per care target person on the basis of the care management information of the care management information storage means.

### [Aspect B4]

Furthermore, the care support system of aspect B4 is the care support system according to aspect B3, further comprising: a target achievement date input means configured to input a target achievement date scheduled to achieve the goal inputted by the goal input means; an update input means configured to input an update of a previous input; an actual fulfillment date input means configured to input a fulfillment date when the desire inputted by the desire input means is actually fulfilled; and an actual achievement date input means configured to input an achievement date when the goal inputted by the goal input means is actually achieved, wherein the care management information registration means registers, in the care management information storage means, the care management information including the identification information inputted by the identification information input means, the desire inputted by the desire input means, the goal inputted by the goal input means, the action item inputted by the action item input means, the degree of achievement inputted by the achievement degree input means, the degree of fulfillment inputted by the fulfillment degree input means, the target achievement date inputted by the target achievement date input means, the update inputted by the update input means, the actual fulfillment date inputted by the actual fulfillment date input means, and the actual achievement date inputted by the actual achievement date input means, and the care management information presenting means presents the identification information, the desire, the goal, the action item, the degree of achievement, the degree of fulfillment, the target achievement date, the update, the actual fulfillment date, and the actual achievement date per care target person on the basis of the care management information of the care management information storage means.

### [Aspect B5]

Furthermore, the care support system of aspect B5 is the care support system according to either aspect B3 or aspect B4, wherein the goal information storage means stores goal information indicating one or more goals for fulfilling the desire of the care target person or his/her family, and the degrees of difficulty in achieving the one or more goals, associating the goal information with the desire, and upon the degree of achievement inputted by the achievement degree input means or the degree of fulfillment inputted by the fulfillment degree input means, both of which are related to the care target person, being equal to or less than a predetermined degree, the goal presenting means presents a goal whose degree of difficulty in achievement corresponding to the goal is equal to or less than a predetermined degree among one or more goals for fulfilling the desire of the care target person inputted by the desire input means, on the basis of the goal information of the goal information storage means.

### [Aspect B6]

Furthermore, the care support system of aspect B6 is the care support system according to either aspect B3 or aspect B4, further comprising a second action item presenting means configured to present an action item in accordance with the degree of achievement inputted by the achievement degree input means, or the degree of fulfillment inputted by the fulfillment degree input means, on the basis of the action item information of the action item information storage means.

### [Aspect B7]

Furthermore, the care support system of aspect B7 is the care support system according to any one of aspects B1 to B4, further comprising a recommended goal presenting means configured to present a goal recommended to the care target person among one or more goals for fulfilling the desire of the care target person inputted by the desire input means on the basis of care track record information, which indicates a care track record of the care target person, of a care track record information storage means where the care track record information is stored, and the goal information of the goal information storage means.

### [Aspect B8]

Furthermore, the care support system of aspect B8 is the care support system according to any one of aspects B1 to B4, further comprising: an attribute information acquisition means configured to acquire attribute information indicating an attribute of a first care target person; and a recommended goal presenting means configured to acquire care track record information indicating a care track record of the care target person, the care track record information corresponding to an attribute having the same or a predetermined relationship with the first care target person, from a care track record information storage means where the care track record information is stored, associated with an attribute of the care target person, on the basis of the attribute information acquired by the attribute information acquisition means, and to present a goal recommended to the first care target person among one or more goals for fulfilling a desire of the first care target person inputted by the desire input means, on the basis of the acquired care track record information and the goal information of the goal information storage means.

### [Aspect B9]

Furthermore, the care support system of aspect B9 is the care support system according to any one of aspects B1 to B4, further comprising: an attribute information acquisition means configured to acquire attribute information indicating an attribute of a first care practitioner; and a recommended goal presenting means configured to acquire care track record information indicating a track record of care implemented by the care practitioner for the care target person, the care track record information corresponding to an attribute having the same or a predetermined relationship with the first care practitioner, from a care track record information storage means where the care track record information is stored, associated with an attribute of the care practitioner, on the basis of the attribute information acquired by the attribute information acquisition means, and to present a goal recommended to the first care practitioner among one or more goals for fulfilling a desire of a care target person for whom care is implemented by the first care practitioner, the desire being inputted by the desire input means, on the basis of the acquired care track record information and the goal information of the goal information storage means.

### [Aspect B10]

Furthermore, the care support system of aspect B10 is the care support system according to either aspect B3 or aspect B4, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or less than a predetermined value, present a goal recommended to the care target person among one or more goals for fulfilling the desire of the care target person inputted by the desire input means, on the basis of the goal information of the goal information storage means.

### [Aspect B11]

Furthermore, the care support system of aspect B11 is the care support system according to either aspect B3 or aspect B4, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or greater than a predetermined value, present a goal recommended to the care target person among one or more goals for fulfilling the desire of the care target person inputted by the desire input means, on the basis of the goal information of the goal information storage means.

### [Aspect B12]

Furthermore, the care support system of aspect B12 is the care support system according to aspect B11, wherein the goal information storage means stores goal information indicating one or more goals for fulfilling the desire of the care target person or his/her family, and the degrees of difficulty in achieving the one or more goals, associating the goal information with the desire, and upon the difference or the rank being equal to or greater than the predetermined value, the recommended goal presenting means presents a goal having a different degree of difficulty in achievement from a previous input by the goal input means on the basis of the goal information of the goal information storage means.

### [Aspect B13]

Furthermore, the care support system of aspect B13 is the care support system according to aspect B4, further comprising a goal achievement determination means configured to acquire, from the care management information storage means, care management information including a goal, an action item, and a target achievement date, which were inputted in the past and include the same combination of a goal and an action item as the goal inputted by the goal input means and the action item inputted by the action item input means, and upon the target achievement date inputted by the target achievement date input means being earlier than a target achievement date included in the acquired care management information, determine that it fails to achieve the goal inputted by the goal input means.

### [Aspect B14]

Furthermore, the care support system of aspect B14 is a care support system that supports a care practitioner in implementing care for a care target person, the care support system comprising: a desire input means configured to input a desire of the care target person or his/her family; an issue presenting means configured to present one or more issues in achieving a goal for fulfilling the desire inputted by the desire input means on the basis of issue information, which indicates one or more issues in achieving the goal for fulfilling the desire, of an issue information storage means where the issue information is stored, associated with the desire; an issue input means configured to input any of the issues presented by the issue presenting means; an action item presenting means configured to present one or more action items necessary to achieve the goal for fulfilling the desire inputted by the desire input means on the basis of action item information, which indicates one or more action items necessary to achieve the goal for fulfilling the desire, of an action item information storage means where the action item information is stored, associated with the desire; an action item input means configured to input any of the action items presented by the action item presenting means; an identification information input means configured to input identification information for identifying the care target person; a care management information registration means configured to register, in a care management information storage means, care management information including the identification information inputted by the identification information input means, the desire inputted by the desire input means, the issue inputted by the issue input means, and the action item inputted by the action item input means; and a care management information presenting means configured to present the identification information, the goal, the issue, and the action item per care target person on the basis of the care management information of the care management information storage means.

### [Aspect B15]

Furthermore, the care support system of aspect B15 is a care support system that supports a care practitioner in implementing care for a care target person, the care support system comprising: a goal input means configured to input a goal for fulfilling a desire of the care target person or his/her family; an issue presenting means configured to present one or more issues in achieving the goal inputted by the goal input means on the basis of issue information, which indicates one or more issues in achieving the goal, of an issue information storage means where the issue information is stored, associated with the goal; an issue input means configured to input any of the issues presented by the issue presenting means; an action item presenting means configured to present one or more action items necessary to achieve the goal inputted by the goal input means on the basis of action item information, which indicates one or more action items necessary to achieve the goal, of an action item information storage means where the action item information is stored, associated with the goal; an action item input means configured to input any of the action items presented by the action item presenting means; an identification information input means configured to input identification information for identifying the care target person; a care management information registration means configured to register, in a care management information storage means, care management information including the identification information inputted by the identification information input means, the goal inputted by the goal input means, the issue inputted by the issue input means, and the action item inputted by the action item input means; and a care management information presenting means configured to present the identification information, the goal, the issue, and the action item per care target person on the basis of the care management information of the care management information storage means.

### [Aspect B16]

Meanwhile, aiming at achieving the above-mentioned object, the care support information registration system of aspect B16 is a care support information registration system that registers information used by a care support system that supports a care practitioner in implementing care for a care target person, the care support information registration system comprising: a care management information acquisition means configured to acquire care management information where a desire of the care target person or his/her family, a goal for fulfilling the desire, and an issue in achieving the goal are associated with each other; a desire extraction means configured to extract the desire from the care management information acquired by the care management information acquisition means; a goal extraction means configured to extract the goal from the care management information acquired by the care management information acquisition means; an issue extraction means configured to extract the issue from the care management information acquired by the care management information acquisition means; a goal information registration means configured to register goal information indicating the goal extracted by the goal extraction means in a goal information storage means, associating the goal information with the desire extracted by the desire extraction means; and an issue information registration means configured to register issue information indicating the issue extracted by the issue extraction means in an issue information storage means, associating the issue information with the goal extracted by the goal extraction means.

### [Aspect B17]

Furthermore, the care support information registration system of aspect B17 is the care support information registration system according to aspect B16, wherein in the care management information, the desire of the care target person or his/her family, the goal for fulfilling the desire, the issue in achieving the goal, and an action item necessary to achieve the goal are associated with each other, and the care support information registration system further comprises: an action item extraction means configured to extract the action item from the care management information acquired by the care management information acquisition means; and an action item information registration means configured to register action item information indicating the action item extracted by the action item extraction means in an action item information storage means, associating the action item information with the goal extracted by the goal extraction means.

### [Aspect B18]

Furthermore, the care support information registration system of aspect B18 is a care support information registration system that registers information used by a care support system that supports a care practitioner in implementing care for a care target person, the care support information registration system comprising: a care management information acquisition means configured to acquire care management information where a desire of the care target person or his/her family, a goal for fulfilling the desire, and an action item necessary to achieve the goal are associated with each other; a desire extraction means configured to extract the desire from the care management information acquired by the care management information acquisition means; a goal extraction means configured to extract the goal from the care management information acquired by the care management information acquisition means; an action item extraction means configured to extract the action item from the care management information acquired by the care management information acquisition means; a goal information registration means configured to register goal information indicating the goal extracted by the goal extraction means in a goal information storage means, associating the goal information with the desire extracted by the desire extraction means; and an action item information registration means configured to register action item information indicating the action item extracted by the action item extraction means in an action item information storage means, associating the action item information with the goal extracted by the goal extraction means.

### [Aspect B19]

Furthermore, the care support information registration system of aspect B19 is a care support information registration system that registers information used by a care support system that supports a care practitioner in implementing care for a care target person, the care support information registration system comprising: a care management information acquisition means configured to acquire care management information where a desire of the care target person or his/her family, an issue in achieving a goal for fulfilling the desire, and an action item necessary to achieve the goal for fulfilling the desire are associated with each other; a desire extraction means configured to extract the desire from the care management information acquired by the care management information acquisition means; an issue extraction means configured to extract the issue from the care management information acquired by the care management information acquisition means; an action item extraction means configured to extract the action item from the care management information acquired by the care management information acquisition means; an issue information registration means configured to register issue information indicating the issue extracted by the issue extraction means in an issue information storage means, associating the issue information with the desire extracted by the desire extraction means; and an action item information registration means configured to register action item information indicating the action item extracted by the action item extraction means in an action item information storage means, associating the action item information with the desire extracted by the desire extraction means.

### [Aspect B20]

Furthermore, the care support information registration system of aspect B20 is a care support information registration system that registers information used by a care support system that supports a care practitioner in implementing care for a care target person, the care support information registration system comprising: a care management information acquisition means configured to acquire care management information where a goal for fulfilling a desire of the care target person or his/her family, an issue in achieving the goal, and an action item necessary to achieve the goal are associated with each other; a goal extraction means configured to extract the goal from the care management information acquired by the care management information acquisition means; an issue extraction means configured to extract the issue from the care management information acquired by the care management information acquisition means; an action item extraction means configured to extract the action item from the care management information acquired by the care management information acquisition means; an issue information registration means configured to register issue information indicating the issue extracted by the issue extraction means in an issue information storage means, associating the issue information with the goal extracted by the goal extraction means; and an action item information registration means configured to register action item information indicating the action item extracted by the action item extraction means in an action item information storage means, associating the action item information with the goal extracted by the goal extraction means.

### [Aspect B21]

Meanwhile, aiming at achieving the above-mentioned object, the care support method of aspect B21 is a care support method for supporting a care practitioner in implementing care for a care target person, the care support method comprising: a desire input step of causing a desire input means to input a desire of the care target person or his/her family; a goal presenting step of causing a goal presenting means to present one or more goals for fulfilling the desire inputted in the desire input step on the basis of goal information, which indicates one or more goals for fulfilling the desire of the care target person or his/her family, of a goal information storage means where the goal information is stored, associated with the desire; a goal input step of causing a goal input means to input any of the goals presented in the goal presenting step; an action item presenting step of causing an action item presenting means to present one or more action items necessary to achieve the goal inputted in the goal input step on the basis of action item information, which indicates one or more action items necessary to achieve the goal related to the goal information, of an action item information storage means where the action item information is stored, associated with the goal; an action item input step of causing an action item input means to input any of the action items presented in the action item presenting step; an identification information input step of causing an identification information input means to input identification information for identifying the care target person; a care management information registration step of causing a care management information registration means to register, in a care management information storage means, care management information including the identification information inputted in the identification information input step, the desire inputted in the desire input step, the goal inputted in the goal input step, and the action item inputted in the action item input step; and a care management information presenting step of causing a care management information presenting means to present the identification information, the desire, the goal, and the action item per care target person on the basis of the care management information of the care management information storage means.

### [Aspect B22]

Furthermore, the care support method of aspect B22 is a care support method for supporting a care practitioner in implementing care for a care target person, the care support method comprising: a desire input step of causing a desire input means to input a desire of the care target person or his/her family; an issue presenting step of causing an issue presenting means to present one or more issues in achieving a goal for fulfilling the desire inputted in the desire input step on the basis of issue information, which indicates one or more issues in achieving the goal for fulfilling the desire, of an issue information storage means where the issue information is stored, associated with the desire; an issue input step of causing an issue input means to input any of the issues presented in the issue presenting step; an action item presenting step of causing an action item presenting means to present one or more action items necessary to achieve the goal for fulfilling the desire inputted in the desire input step on the basis of action item information, which indicates one or more action items necessary to achieve the goal for fulfilling the desire, of an action item information storage means where the action item information is stored, associated with the desire; an action item input step of causing an action item input means to input any of the action items presented in the action item presenting step; an identification information input step of causing an identification information input means to input identification information for identifying the care target person; a care management information registration step of causing a care management information registration means to register, in a care management information storage means, care management information including the identification information inputted in the identification information input step, the desire inputted in the desire input step, the issue inputted in the issue input step, and the action item inputted in the action item input step; and a care management information presenting step of causing a care management information presenting means to present the identification information, the desire, the issue, and the action item per care target person on the basis of the care management information of the care management information storage means.

### [Aspect B23]

Furthermore, the care support method of aspect B23 is a care support method for supporting a care practitioner in implementing care for a care target person, the care support method comprising: a goal input step of causing a goal input means to input a goal for fulfilling a desire of the care target person or his/her family; an issue presenting step of causing an issue presenting means to present one or more issues in achieving the goal inputted in the goal input step on the basis of issue information, which indicates one or more issues in achieving the goal, of an issue information storage means where the issue information is stored, associated with the goal; an issue input step of causing an issue input means to input any of the issues presented in the issue presenting step; an action item presenting step of causing an action item presenting means to present one or more action items necessary to achieve the goal inputted in the goal input step on the basis of action item information, which indicates one or more action items necessary to achieve the goal, of an action item information storage means where the action item information is stored, associated with the goal; an action item input step of causing an action item input means to input any of the action items presented in the action item presenting step; an identification information input step of causing an identification information input means to input identification information for identifying the care target person; a care management information registration step of causing a care management information registration means to register, in a care management information storage means, care management information including the identification information inputted in the identification information input step, the goal inputted in the goal input step, the issue inputted in the issue input step, and the action item inputted in the action item input step; and a care management information presenting step of causing a care management information presenting means to present the identification information, the goal, the issue, and the action item per care target person on the basis of the care management information of the care management information storage means.

### [Aspect B24]

Meanwhile, aiming at achieving the above-mentioned object, the care support information registration method of aspect B24 is a care support information registration method for registering information used by a care support system that supports a care practitioner in implementing care for a care target person, the care support information registration method comprising: a care management information acquisition step of causing a care management information acquisition means to acquire care management information where a desire of the care target person or his/her family, a goal for fulfilling the desire, and an issue in achieving the goal are associated with each other; a desire extraction step of causing a desire extraction means to extract the desire from the care management information acquired in the care management information acquisition step; a goal extraction step of causing a goal extraction means to extract the goal from the care management information acquired in the care management information acquisition step; an issue extraction step of causing an issue extraction means to extract the issue from the care management information acquired in the care management information acquisition step; a goal information registration step of causing a goal information registration means to register goal information indicating the goal extracted in the goal extraction step in a goal information storage means, associating the goal information with the desire extracted in the desire extraction step; and an issue information registration step of causing an issue information registration means to register issue information indicating the issue extracted in the issue extraction step in an issue information storage means, associating the issue information with the goal extracted in the goal extraction step.

### [Aspect B25]

Furthermore, the care support information registration method of aspect B25 is a care support information registration method for registering information used by a care support system that supports a care practitioner in implementing care for a care target person, the care support information registration method comprising: a care management information acquisition step of causing a care management information acquisition means to acquire care management information where a desire of the care target person or his/her family, a goal for fulfilling the desire, and an action item necessary to achieve the goal are associated with each other; a desire extraction step of causing a desire extraction means to extract the desire from the care management information acquired in the care management information acquisition step; a goal extraction step of causing a goal extraction means to extract the goal from the care management information acquired in the care management information acquisition step; an action item extraction step of causing an action item extraction means to extract the action item from the care management information acquired in the care management information acquisition step; a goal information registration step of causing a goal information registration means to register goal information indicating the goal extracted in the goal extraction step in a goal information storage means, associating the goal information with the desire extracted in the desire extraction step; and an action item information registration step of causing an action item information registration means to register action item information indicating the action item extracted in the action item extraction step in an action item information storage means, associating the action item information with the goal extracted in the goal extraction step.

### [Aspect B26]

Furthermore, the care support information registration method of aspect B26 is a care support information registration method for registering information used by a care support system that supports a care practitioner in implementing care for a care target person, the care support information registration method comprising: a care management information acquisition step of causing a care management information acquisition means to acquire care management information where a desire of the care target person or his/her family, an issue in achieving a goal for fulfilling the desire, and an action item necessary to achieve the goal for fulfilling the desire are associated with each other; a desire extraction step of causing a desire extraction means to extract the desire from the care management information acquired in the care management information acquisition step; an issue extraction step of causing an issue extraction means to extract the issue from the care management information acquired in the care management information acquisition step; an action item extraction step of causing an action item extraction means to extract the action item from the care management information acquired in the care management information acquisition step; an issue information registration step of causing an issue information registration means to register issue information indicating the issue extracted in the issue extraction step in an issue information storage means, associating the issue information with the desire extracted in the desire extraction step; and an action item information registration step of causing an action item information registration means to register action item information indicating the action item extracted in the action item extraction step in an action item information storage means, associating the action item information with the desire extracted in the desire extraction step.

### [Aspect B27]

Furthermore, the care support information registration method of aspect B27 is a care support information registration method for registering information used by a care support system that supports a care practitioner in implementing care for a care target person, the care support information registration method comprising: a care management information acquisition step of causing a care management information acquisition means to acquire care management information where a goal for fulfilling a desire of the care target person or his/her family, an issue in achieving the goal, and an action item necessary to achieve the goal are associated with each other; a goal extraction step of causing a goal extraction means to extract the goal from the care management information acquired in the care management information acquisition step; an issue extraction step of causing an issue extraction means to extract the issue from the care management information acquired in the care management information acquisition step; an action item extraction step of causing an action item extraction means to extract the action item from the care management information acquired in the care management information acquisition step; an issue information registration step of causing an issue information registration means to register issue information indicating the issue extracted in the issue extraction step in an issue information storage means, associating the issue information with the goal extracted in the goal extraction step; and an action item information registration step of causing an action item information registration means to register action item information indicating the action item extracted in the action item extraction step in an action item information storage means, associating the action item information with the goal extracted in the goal extraction step.

### Effects of Invention

As described above, according to the care support system of aspect A1, or the care support method of aspect A12, one or more goals for fulfilling the desire of the care target person or his/her family are presented, and the care practitioner is simply required to select any of the goals. Furthermore, one or more issues in achieving the selected goal are presented, and the care practitioner is simply required to select any of the issues. Accordingly, even a care practitioner with little experience can create a care plan for fulfilling the desire of the care target person or his/her family compared to before. Moreover, it is possible to grasp the identification information, the desire, the goal, and the issue per care target person. Accordingly, it is possible to implement care for fulfilling the desire of the care target person or his/her family.

Furthermore, according to the care support system of aspect A2, it is possible to grasp the identification information, the desire, the goal, the issue, the degree of achievement, and the degree of fulfillment per care target person. Accordingly, it is possible to more appropriately implement care for fulfilling the desire of the care target person or his/her family.

Furthermore, according to the care support system of aspect A3, it is possible to grasp the identification information, the desire, the goal, the issue, the degree of achievement, the degree of fulfillment, the target achievement date, the update, the actual fulfillment date, and the actual achievement date, per care target person. Accordingly, it is possible to more appropriately implement care for fulfilling the desire of the care target person or his/her family.

Furthermore, according to the care support system of aspect A4, if the degree of achievement or the degree of fulfillment is equal to or less than the predetermined degree, a goal whose degree of difficulty in achievement is equal to or less than the predetermined degree is presented. Accordingly, it is possible to implement care in such a manner as to near the fulfillment of the desire of the care target person or his/her family.

Furthermore, according to the care support system of aspect A5, an action item is presented in accordance with the degree of achievement or the degree of fulfillment. Accordingly, it is possible to implement care in such a manner as to near the fulfillment of the desire of the care target person or his/her family.

Furthermore, according to the care support system of aspect A6, a goal recommended to the care target person is presented in accordance with the past care track record of the care target person. Accordingly, it is possible to create a care plan that is effective in fulfilling the desire of the care target person or his/her family.

Furthermore, according to the care support system of aspect A7, a goal recommended to the first care target person is presented in accordance with the past care track record of the care target person of the attribute having the same or predetermined relationship with the first care target person. Accordingly, it is possible to create a care plan that is effective in fulfilling the desire of the first care target person or his/her family.

Furthermore, according to the care support system of aspect A8, a goal recommended to the care target person for whom care is implemented by the first care practitioner is presented in accordance with the track record of the past care implemented by the care practitioner of the attribute having the same or predetermined relationship with the first care practitioner. Accordingly, it is possible to create a care plan that is effective in fulfilling the desire of the care target person or his/her family.

Furthermore, according to the care support system of aspect A9, if the difference between the degree of achievement and the degree of fulfillment, or the rank thereof is equal to or less than, or equal to or greater than, the predetermined value, a goal recommended to the care target person is presented. Accordingly, it is possible to implement care in such a manner as to near the fulfillment of the desire of the care target person or his/her family.

Furthermore, according to the care support system of aspect A10, if the difference between the degree of achievement and the degree of fulfillment, or the rank thereof is equal to or greater than the predetermined value, a goal having a different degree of difficulty in achievement from the previous input is presented. Accordingly, it is possible to implement care in such a manner as to near the fulfillment of the desire of the care target person or his/her family.

Furthermore, according to the care support system of aspect A11, whether or not the goal can be achieved is determined from the past track record and the inputted information. Accordingly, it is possible to prevent the creation of a care plan having a low possibility of fulfilling the desire of the care target person or his/her family.

Furthermore, according to the care support system of aspect B1, or the care support method of aspect B21, one or more goals for fulfilling the desire of the care target person or his/her family are presented. The care practitioner is simply required to select any of the goals. Furthermore, one or more action items necessary to achieve the selected goal are presented. The care practitioner is simply required to select any of the action items. Accordingly, even a care practitioner with little experience can create a care plan for fulfilling the desire of the care target person or his/her family. Moreover, it is possible to grasp the identification information, the desire, the goal, and the action item per care target person. Accordingly, it is possible to implement care for fulfilling the desire of the care target person or his/her family.

Furthermore, according to the care support system of aspect B2, it is possible to grasp the identification information, the desire, the goal, the issue, and the action item per care target person. Accordingly, it is possible to implement care for fulling the desire of the care target person or his/her family.

Furthermore, according to the care support system of aspect B3, it is possible to grasp the identification information, the desire, the goal, the action item, the degree of achievement, and the degree of fulfillment per care target person. Accordingly, it is possible to more appropriately implement care for fulfilling the desire of the care target person or his/her family.

Furthermore, according to the care support system of aspect B4, it is possible to grasp the identification information, the desire, the goal, the action item, the degree of achievement, the degree of fulfillment, the target achievement date, the update, the actual fulfillment date, and the actual achievement date per care target person. Accordingly, it is possible to more appropriately implement care for fulfilling the desire of the care target person or his/her family.

Furthermore, according to the care support system of aspect B5, if the degree of achievement or the degree of fulfillment is equal to or less than the predetermined degree, a goal whose degree of difficulty in achievement is equal to or less than the predetermined degree is presented. Accordingly, it is possible to implement care in such a manner as to near the fulfillment of the desire of the care target person or his/her family.

Furthermore, according to the care support system of aspect B6, an action item is presented in accordance with the degree of achievement or the degree of fulfillment. Accordingly, it is possible to implement care in such a manner as to near the fulfillment of the desire of the care target person or his/her family.

Furthermore, according to the care support system of aspect B7, a goal recommended to the care target person is presented in accordance with the past care track record of the care target person. Accordingly, it is possible to create a care plan that is effective in fulfilling the desire of the care target person or his/her family.

Furthermore, according to the care support system of aspect B8, a goal recommended to the first care target person is presented in accordance with the past care track record of the care target person of the attribute having the same or predetermined relationship with the first care target person. Accordingly, it is possible to create a care plan that is effective in fulfilling the desire of the first care target person or his/her family.

Furthermore, according to the care support system of aspect B9, a goal recommended to the care target person for whom care is implemented by the first care practitioner is presented in accordance with the track record of the past care implemented by the care practitioner of the attribute having the same or predetermined relationship with the first care practitioner. Accordingly, it is possible to create a care plan that is effective in fulfilling the desire of the care target person or his/her family.

Furthermore, according to the care support system of aspect B10, if the difference between the degree of achievement and the degree of fulfillment, or the rank thereof is equal to or less than the predetermined degree, a goal recommended to the care target person is presented. Accordingly, it is possible to implement care in such a manner as to near the fulfillment of a desire of the care target person or his/her family.

Furthermore, according to the care support system of aspect B11, if the difference between the degree of achievement and the degree of fulfillment, or the rank thereof is equal to or greater than the predetermined value, a goal recommended to the care target person is presented. Accordingly, it is possible to implement care in such a manner as to near the fulfillment of the desire of the care target person or his/her family.

Furthermore, according to the care support system of aspect B12, if the difference between the degree of achievement and the degree of fulfillment, or the rank thereof is equal to or greater than the predetermined degree, a goal having a different degree of difficulty in achievement from the previous input is presented. Accordingly, it is possible to implement care in such a manner as to near the fulfillment of the desire of the care target person or his/her family.

Furthermore, according to the care support system of aspect B13, whether or not the goal can be achieved is determined from the past track record and the inputted information. Accordingly, it is possible to prevent the creation of a care plan having a low possibility of fulfilling the desire of the care target person or his/her family.

Furthermore, according to the care support system of aspect B14, or the care support method of aspect B22, one or more issues in achieving the goal for fulfilling the desire of the care target person or his/her family are presented. The care practitioner is simply required to select any of the issues. Furthermore, one or more action items necessary to achieve the goal for fulfilling the desire of the care target person or his/her family are presented. The care practitioner is simply required to select any of the action items. Accordingly, even a care practitioner with little experience can create a care plan for fulfilling the desire of the care target person or his/her family compared to before. Moreover, it is possible to grasp the identification information, the desire, the issue, and the action item per care target person. Accordingly, it is possible to implement care for fulfilling the desire of the care target person or his/her family.

Furthermore, according to the care support system of aspect B15, or the care support method of aspect B23, one or more issues in achieving the goal for fulfilling the desire of the care target person or his/her family are presented. The care practitioner is simply required to select any of the issues. Furthermore, one or more action items necessary to achieve the goal for fulfilling the desire of the care target person or his/her family are presented. The care practitioner is simply required to select any of the action items. Accordingly, even a care practitioner with little experience can create a care plan for fulfilling the desire of the care target person or his/her family compared to before. Moreover, it is possible to grasp the identification information, the goal, the issue, and the action item per care target person. Accordingly, it is possible to implement care for fulfilling the desire of the care target person or his/her family.

On the other hand, according to the care support information registration system of aspect B16, or the care support information registration method of aspect B24, it is possible to obtain the goal information associated with the desire and the issue information associated with the goal. These pieces of information can be used in, for example, a care support system of Japanese Patent Application No. 2019-94484.

Furthermore, according to the care support information registration system of aspect B17, it is possible to obtain the goal information associated with the desire, the issue information associated with the goal, and the action item information associated with the goal. These pieces of information can be used in, for example, the care support system of aspect B2.

Furthermore, according to the care support information registration system of aspect B18, or the care support information registration method of aspect B25, it is possible to obtain the goal information associated with the desire and the action item information associated with the goal. These pieces of information can be used in, for example, the care support system of aspect B1.

Furthermore, according to the care support information registration system of aspect B19, or the care support information registration method of aspect B26, it is possible to obtain the issue information associated with the desire and the action item information associated with the desire. These pieces of information can be used in, for example, the care support system of aspect B14.

Furthermore, according to the care support information registration system of aspect B20, or the care support information registration method of aspect B27, it is possible to obtain the issue information associated with the goal and the action item information associated with the goal. These pieces of information can be used in, for example, the care support system of aspect B15.

### BRIEF DESCRIPTION OF DRAWINGS

FIG. 1 is a diagram illustrating the hardware configuration of a care support apparatus 100.

FIG. 2 is a conceptual diagram illustrating data coupling relationships of tables 400 to 440.

FIG. 3 is a diagram illustrating the data structure of the desire information table 400.

FIG. 4 is a diagram illustrating the data structure of the goal information table 420.

FIG. 5 is a diagram illustrating the data structure of the issue information table 440.

FIG. 6 is a flowchart illustrating a care support process.

FIG. 7 is a screen illustrating a care management table in a case of inputting a desire from a plurality of candidates.

FIG. 8 is a screen illustrating a care management table in a case of inputting a goal from a plurality of candidates.

FIG. 9 is a screen illustrating a care management table in a case of inputting an issue from a plurality of candidates.

FIG. 10 is a conceptual diagram illustrating data coupling relationships of tables 400 to 460.

FIG. 11 is a diagram illustrating the data structure of the desire information table 400.

FIG. 12 is a diagram illustrating the data structure of the goal information table 420.

FIG. 13 is a diagram illustrating the data structure of the issue information table 440.

FIG. 14 is a diagram illustrating the data structure of the action item information table 460.

FIG. 15 is a flowchart illustrating a care support process.

FIG. 16 is a screen illustrating a care management table in a case of inputting a desire from a plurality of candidates.

FIG. 17 is a screen illustrating a care management table in a case of inputting a goal from a plurality of candidates.

FIG. 18 is a screen illustrating a care management table in a case of inputting an issue from a plurality of candidates.

FIG. 19 is a screen illustrating a care management table in a case of inputting an action item from a plurality of candidates.

FIG. 20 is a flowchart illustrating a care support information registration process.

FIG. 21 is a conceptual diagram illustrating data coupling relationships of the tables 420, 440, and 460.

FIG. 22 is a conceptual diagram illustrating data coupling relationships of the tables 400, 420, and 460.

FIG. 23 is a conceptual diagram illustrating data coupling relationships of the tables 400, 440, and 460.

FIG. 24 is a conceptual diagram illustrating data coupling relationships of the tables 400, 420, and 440.

### DESCRIPTION OF EMBODIMENTS

### First Embodiment

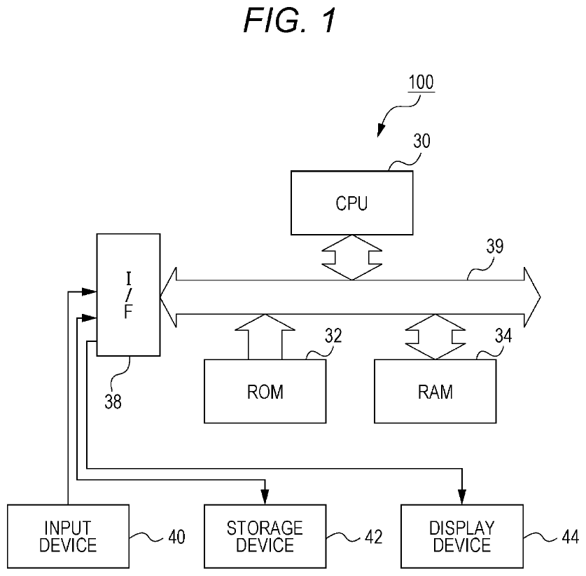

A first embodiment of the present invention is described below. FIGS. 1 to 9 are diagrams illustrating the embodiment.

Firstly, the configuration of the embodiment is described.

FIG. 1 is a diagram illustrating the hardware configuration of a care support apparatus 100.

As illustrated in FIG. 1, the care support apparatus 100 includes a CPU (Central Processing Unit) 30 that performs computations and controls the entire system on the basis of a control program, a ROM (Read Only Memory) 32 that stores in advance, for example, the control program of the CPU 30 in a predetermined area, a RAM (Random Access Memory) 34 for storing data read from, for example, the ROM 32, and computation results necessary in the course of computation of the CPU 30, and an I/F (InterFace) 38 through which data is inputted into and outputted from an external device. They are connected to each other with a bus 39 being a signal line for transferring data in such a manner as to be able to exchange data.

The I/F 38 is connected to, as external devices, an input device 40 including a keyboard and a mouse, which can input data as human interfaces, a storage device 42 that stores, for example, data and tables as files, and a display device 44 that displays screens on the basis of image signals.

Next, the data structure of the storage device 42 is described.

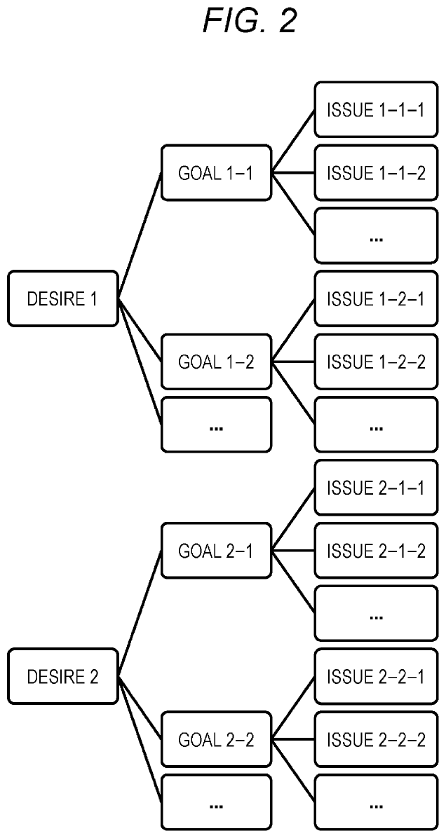

FIG. 2 is a conceptual diagram illustrating the data coupling relationship of tables 400 to 440.

Care practitioners, mainly care practitioners with rich experience, list in advance (1) candidates for a desire of a care target person, (2) candidates for a goal for fulfilling the desire, and (3) candidates for an issue (a problem or a point to be improved) in achieving the goal.

As illustrated in FIG. 2, for one desire, one or more goals for fulfilling the desire can be set. In the example of FIG. 2, a goal 1-1, a goal 1-2, and other goals are set for a desire 1. A goal 2-1, a goal 2-2, and other goals are set for a desire 2. Each desire is associated with goals to be set. Accordingly, the tendency and the content may be common or similar in the goals 1-1, 1-2, 2-1, and 2-2. However, the goals 1-1, 1-2, 2-1, and 2-2 are basically independent of each other.

Next, for one goal, one or more issues in achieving the goal can be set. In the example of FIG. 2, an issue 1-1-1, an issue 1-1-2, and other issues are set for the goal 1-1. An issue 1-2-1, an issue 1-2-2, and other issues are set for the goal 1-2. An issue 2-1-1, an issue 2-1-2, and other issues are set for the goal 2-1. An issue 2-2-1, an issue 2-2-2, and other issues are set for the goal 2-2. Each goal is associated with issues to be set. Accordingly, the tendency and the content may be common or similar in the issues 1-1-1, 1-1-2, 1-2-1, 1-2-2, 2-1-1, 2-1-2, 2-2-1, and 2-2-2. However, the issues 1-1-1, 1-1-2, 1-2-1, 1-2-2, 2-1-1, 2-1-2, 2-2-1, and 2-2-2 are basically independent of each other.

This data structure is realized by the desire information table 400, the goal information table 420, and the issue information table 440.

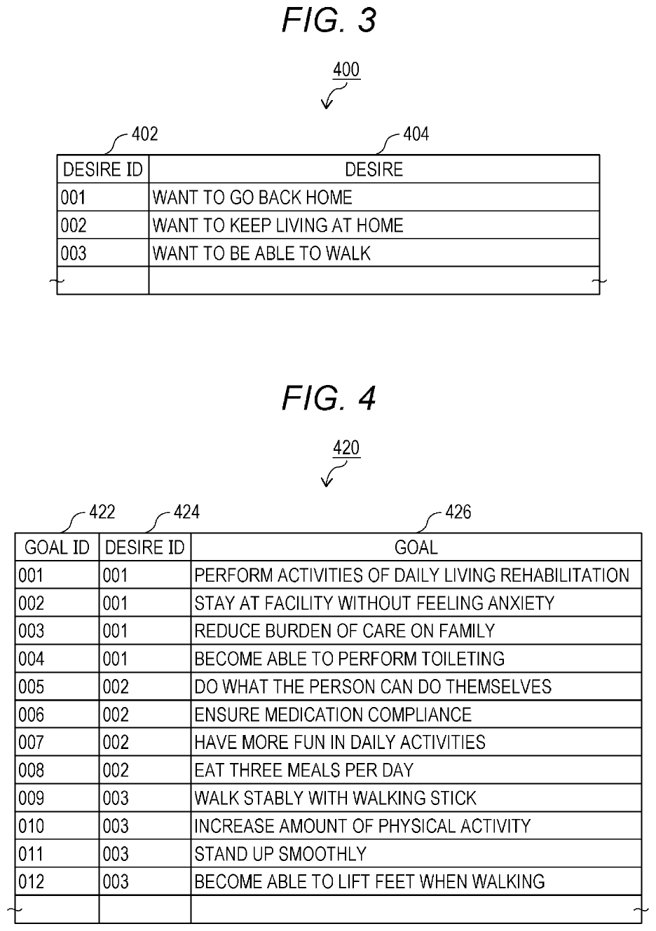

FIG. 3 is a diagram illustrating the data structure of the desire information table 400.

As illustrated in FIG. 3, the desire information table 400 is stored in the storage device 42.

One record is registered per desire in the desire information table 400. Each record is configured including a field 402 where a desire ID for identifying a desire is registered, and a field 404 where the desire (in text) is registered. In the example of FIG. 3, “001” is registered as the desire ID, and “want to go back home” as the desire in a record in the first row. This indicates that the desire identified by the desire ID “001” is “want to go back home.”

FIG. 4 is a diagram illustrating the data structure of the goal information table 420.

As illustrated in FIG. 4, the goal information table 420 is stored in the storage device 42.

One record is registered per goal in the goal information table 420. Each record is configured including a field 422 where a goal ID for identifying a goal is registered, a field 424 where a desire ID of a desire attempted to be fulfilled by achieving the goal is registered, and a field 426 where the goal (in text) is registered. In the example of FIG. 4, “001” is registered as the goal ID, “001” as the desire ID, and “perform activities of daily living rehabilitation” as the goal in a record in the first row. This indicates that the goal identified by the goal ID “001” is “perform activities of daily living rehabilitation” and is set as the goal for fulfilling the desire “want go back home” of the desire ID “001”.

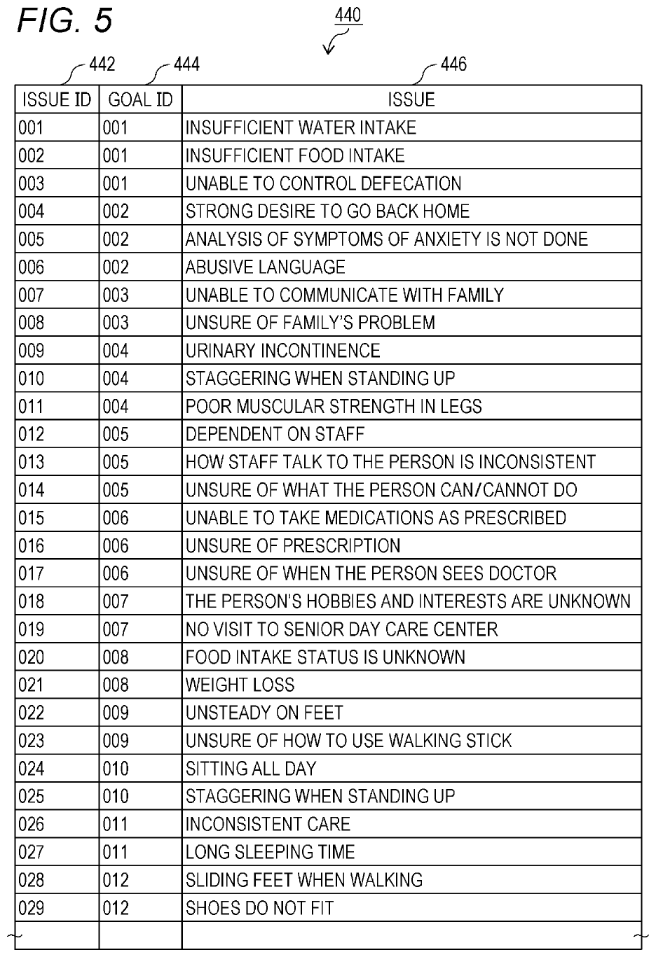

FIG. 5 is a diagram illustrating the data structure of the issue information table 440.

As illustrated in FIG. 5, the issue information table 440 is stored in the storage device 42.

One record is registered per issue in the issue information table 440. Each record is configured including a field 442 where an issue ID for identifying an issue is registered, a field 444 where a goal ID of a goal attempted to be achieved by solving the issue is registered, and a field 446 where the issue (in text) is registered. In the example of FIG. 5, “001” is registered as the issue ID, “001” as the goal ID, and “insufficient water intake” as the issue in a record in the first row. This indicates that the issue identified by the issue ID “001” is “insufficient water intake” and is set as the issue in achieving the goal “perform activities of daily living rehabilitation” of the goal ID “001”.

Next, the operation of the embodiment is described.

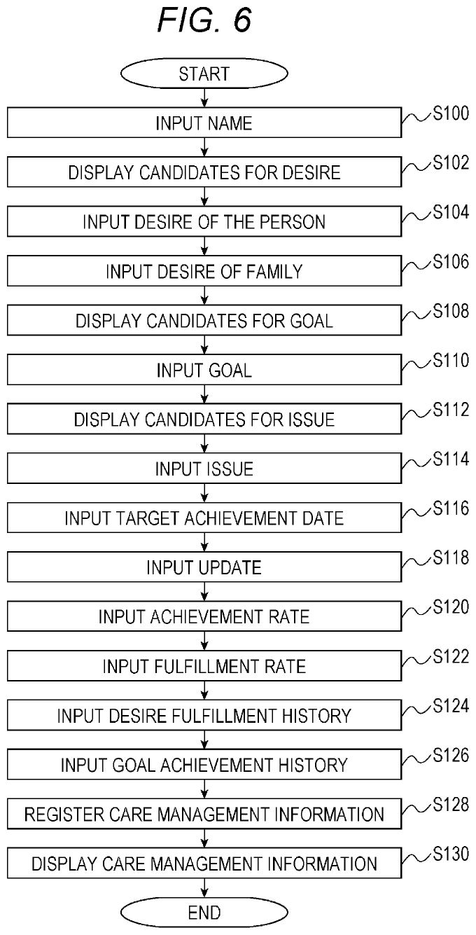

The CPU 30 includes, for example, an MPU (Micro-Processing Unit). The CPU 30 starts a predetermined program stored in a predetermined area of the ROM 32, and executes a care support process illustrated in a flowchart of FIG. 6 in accordance with the program.

FIG. 6 is a flowchart illustrating the care support process.

The care support process is a process that is executed repeatedly at predetermined intervals. When the CPU 30 executes the care support process, the procedure moves on to step S100 first as illustrated in FIG. 6.

In step S100, if the input of the name of a care target person is requested, the name of the care target person is inputted through the input device 40.

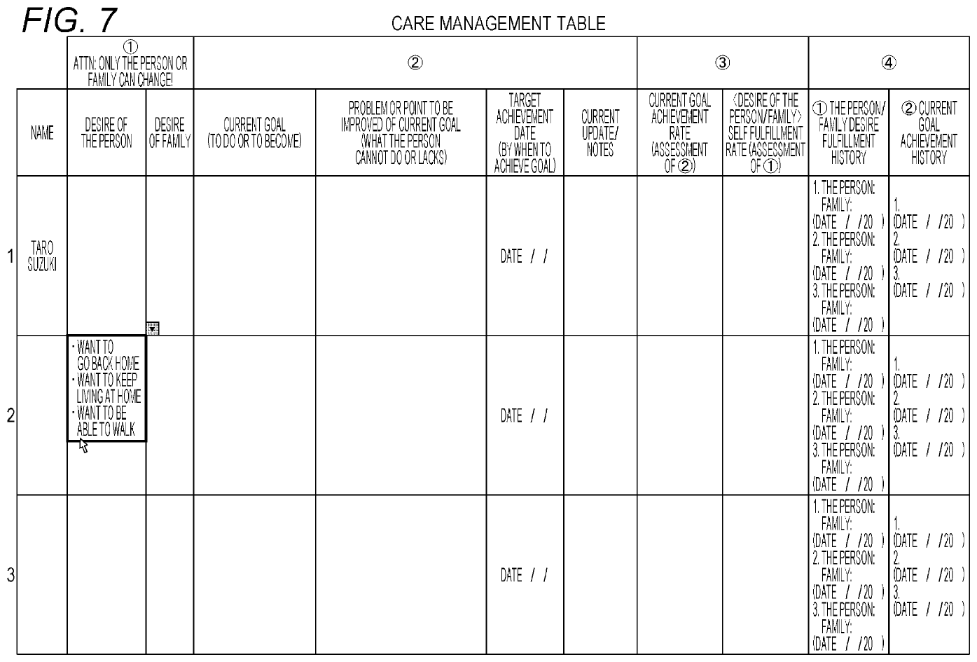

FIG. 7 is a screen illustrating a care management table in a case of inputting a desire from a plurality of candidates.

Next, the procedure moves on to step S102. If the input of a desire of the care target person is requested, one or more desires are read from the desire information table 400. As illustrated in FIG. 7, candidates for the desire of the care target person are displayed on the display device 44. A care practitioner selects any of the displayed candidates. In the example of FIG. 7, “want to go back home”, “want to keep living at home”, “want to be able to walk” are displayed as the candidates for the desire. It is assumed that the care practitioner selects, for example, “want to go back home”. If the desire is selected, the procedure moves on to step S104. The selected desire is inputted through the input device 40.

Next, the procedure moves on to step S106. If the input of a desire of a family of the care target person is requested, the desire of the family of the care target person is inputted through the input device 40.

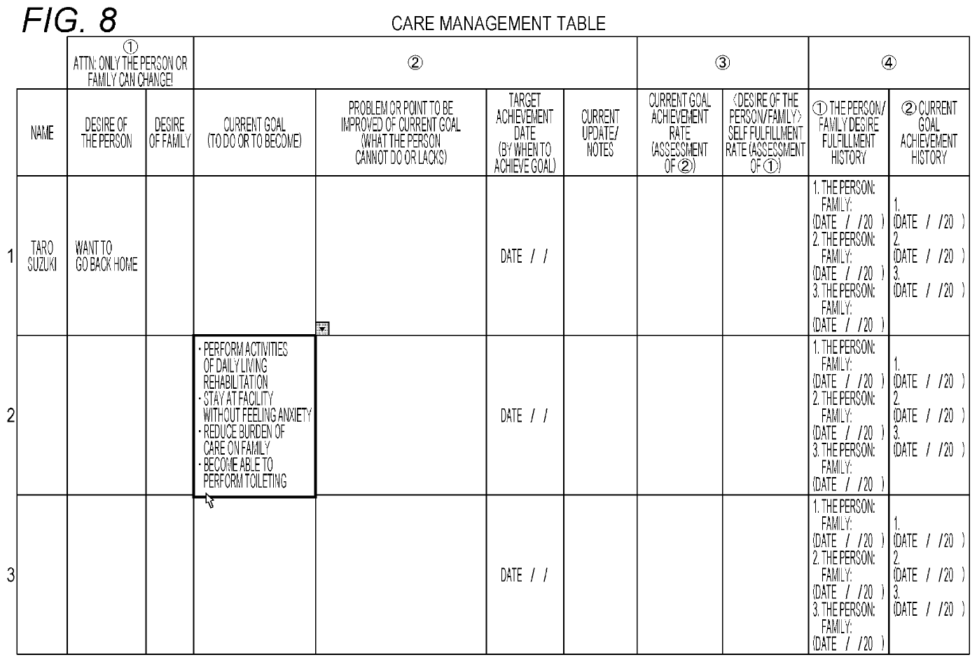

FIG. 8 is a screen illustrating a care management table in a case of inputting a goal from a plurality of candidates.

Next, the procedure moves on to step S108. If the input of a goal is requested, one or more goals corresponding to a desire ID of the desire inputted in step S104 are read from the goal information table 420. As illustrated in FIG. 8, candidates for a goal for fulfilling the desire are displayed on the display device 44. The care practitioner selects any of the displayed candidates. In the example of FIG. 8, “perform activities of daily living rehabilitation”, “stay at a facility without feeling anxiety”, “reduce the burden of care on the family”, and “become able to perform toileting” are displayed as the candidates for the goal. It is assumed that the care practitioner selects, for example, “perform activities of daily living rehabilitation”. If the goal is selected, the procedure moves on to step S110. The selected goal is inputted through the input device 40.

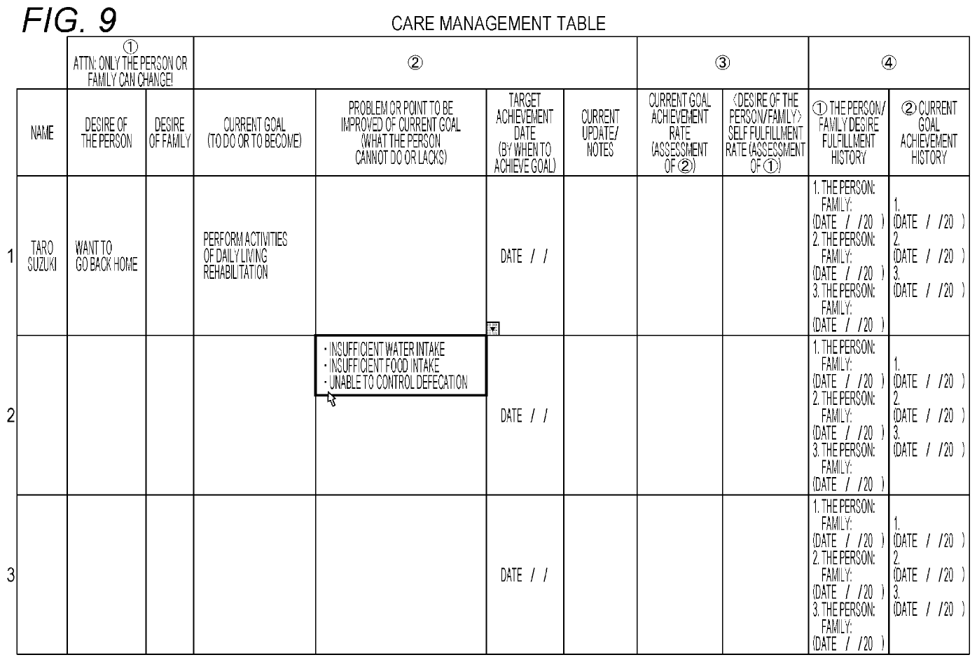

FIG. 9 is a screen illustrating a care management table in a case of inputting an issue from a plurality of candidates.

Next, the procedure moves on to step S112. If the input of an issue is requested, one or more issues corresponding to a goal ID of the goal inputted in step S110 are read from the issue information table 420. As illustrated in FIG. 9, candidates for an issue in achieving the goal are displayed on the display device 44. The care practitioner selects any of the displayed candidates. In the example of FIG. 8, “perform activities of daily living rehabilitation”, “stay at a facility without feeling anxiety”, “reduce the burden of care on the family”, and “become able to perform toileting” are displayed as the candidates for the issue. It is assumed that the care practitioner selects, for example, “perform activities of daily living rehabilitation”. If the issue is selected, the procedure moves on to step S114. The selected issue is inputted through the input device 40.

Next, the procedure moves on to step S116. If the input of a target achievement date is requested, a target achievement date scheduled to achieve the goal is inputted through the input device 40.

Next, the procedure moves on to step S118. If the input of an update is requested, an update of a previous input is inputted through the input device 40.

Next, the procedure moves on to step S120. If the input of an achievement rate is requested, the achievement rate of the goal (in percentage) is inputted through the input device 40.

Next, the procedure moves on to step S122. If the input of a fulfillment rate is requested, the fulfillment rate of the desire (in percentage) is inputted through the input device 40. The fulfillment rate is inputted on the basis of an answer from the care target person or family.

Next, the procedure moves on to step S124. If the input of a desire fulfillment history is requested, a desire fulfillment history including the date when the desire came true is inputted through the input device 40. The desire fulfillment history is inputted on the basis of an answer form the care target person or family.

Next, the procedure moves on to step S126. If the input of a goal achievement history is requested, a goal achievement history including the date when the goal was achieved is inputted through the input device 40.

Next, the procedure moves on to step S128. Care management information including the name, the desire of the care target person, the desire of the family, the goal, the issue, the target achievement date, the update, the achievement rate, the fulfillment rate, the desire fulfillment history, and the goal achievement history, which were inputted in steps S100, S104, S106, S110, S114, and S116 to S126, is registered in the storage device 42. The care management information is registered per care target person.

The procedure then moves on to step S130. The name, the desire of the care target person, the desire of the family, the goal, the issue, the target achievement date, the update, the achievement rate, the fulfillment rate, the desire fulfillment history, and the goal achievement history are displayed per care target person on the display device 44 on the basis of the care management information of the storage device 42. For example, items corresponding to the care management information are displayed in the fields of the care management table of FIGS. 7 to 9, respectively.

Next, the effects of the embodiment are described.

In the embodiment, a desire of a care target person is inputted. Candidates for a goal for fulfilling the inputted desire are displayed on the basis of the goal information table 420. Any of the displayed goals is inputted. One or more issues in achieving the inputted goal are displayed on the basis of the issue information table 440. Any of the displayed issues is inputted. Care management information including the name of the care target person, and the inputted desire, goal, and issue is registered in the storage device 42. The name, the desire, the goal, and the issue are displayed per care target person on the basis of the care management information of the storage device 42.

Consequently, one or more goals for fulfilling the desire of the care target person are displayed, and a care practitioner is simply required to select any of the goals. Furthermore, one or more issues in achieving the selected goal are displayed, and the care practitioner is simply required to select any of the issues. Accordingly, even if a care practitioner with little experience can create a care plan for fulfilling the desire of the care target person compared to before. Moreover, it is possible to grasp the name, the desire, the goal, and the issue per care target person. Accordingly, it is possible to implement care for fulfilling the desire of the care target person.

Furthermore, in the embodiment, an achievement rate of the inputted goal is inputted. A fulfillment rate of the inputted desire is inputted. Care management information including the name of the care target person, and the inputted desire, goal, issue, achievement rate, and fulfillment rate is registered in the storage device 42. The name, the desire, the goal, the issue, the achievement rate, and the fulfillment rate are displayed per care target person on the basis of the care management information of the storage device 42.

Consequently, it is possible to grasp the name, the desire, the goal, the issue, the achievement rate, and the fulfillment rate per care target person. Accordingly, it is possible to more appropriately implement care for fulfilling the desire of the care target person.

Furthermore, in the embodiment, a target achievement date scheduled to achieve the inputted goal is inputted. An update of a previous input is inputted. A desire fulfillment history including the date when the inputted desire came true is inputted. A goal achievement history including the date when the inputted goal was achieved is inputted. Care management information including the name of the care target person, and the inputted desire, goal, issue, achievement rate, fulfillment rate, target achievement date, update, desire fulfillment history, and goal achievement history is registered in the storage device 42. The name, the desire, the goal, the issue, the achievement rate, the fulfillment rate, the target achievement date, the update, the desire fulfillment history, and the goal achievement history are displayed per care target person on the basis of the care management information of the storage device 42.

Consequently, it is possible to grasp the name, the desire, the goal, the issue, the achievement rate, the fulfillment rate, the target achievement date, the update, the desire fulfillment history, and the goal achievement history per care target person. Accordingly, it is possible to more appropriately implement care for fulfilling the desire of the care target person.

In the embodiment, the storage device 42 corresponds to the goal information storage means of aspect A1 or A12, the issue information storage means of aspect A1 or A12, or the care management information storage means of any of aspects A1 to A3 and A12. Step S104 corresponds to the desire input means of any of aspects A1 to A3 and A12, or the desire input step of aspect A12. Moreover, step S108 corresponds to the goal presenting means of aspect A1 or A12, or the goal presenting step of aspect A12. Step S110 corresponds to the goal input means of any of aspects A1 to A3 and A12, or the goal input step of aspect A12. Step S112 corresponds to the issue presenting means of aspect A1 or A12, or the issue presenting step of aspect A12.

Moreover, in the embodiment, step S114 corresponds to the issue input means of any of aspects A1 to A3 and A12, or the issue input step of aspect A12. Step S116 corresponds to the target achievement date input means of aspect A3. Step S118 corresponds to the update input means of aspect A3. Moreover, step S120 corresponds to the achievement degree input means of aspect A2 or A3. Step S122 corresponds to the fulfillment degree input means of aspect A2 or A3. Step S124 corresponds to the actual fulfillment date input means of aspect A3. Step S126 corresponds to the actual achievement date input means of aspect A3.

Moreover, in the embodiment, step S128 corresponds to the care management information registration means of any of aspects A1 to A3 and A12, or the care management information registration step of aspect A12. Step S130 corresponds to the care management information presenting means of any of aspects A1 to A3 and A12, or the care management information presenting step of aspect A12.

### [Modifications]

The following configurations can be employed as modifications of the above first embodiment.

### [Configuration 1]

This configuration is a configuration that narrows down candidates for a goal in accordance with the achievement rate or fulfillment rate. One or more goals for fulfilling a desire of a care target person, and the degrees of difficulty in achieving the one or more goals are registered, associated with the desire, in the goal information table 420. In the example of FIG. 4, the goal information table 420 further includes a field where the degree of difficulty in achieving a goal is registered. In step S108, if a previous achievement rate inputted in step S120 or a previous fulfillment rate inputted in step S122, both of which are related to a care target person A, is equal to or less than a predetermined value, a goal whose degree of difficulty in achievement is equal to or less than a predetermined degree among one or more goals for fulfilling the desire of the care target person A inputted in step S104 is displayed on the display device 44 on the basis of the goal information table 420.

Consequently, if the achievement rate or fulfillment rate is equal to or less than the predetermined value, a goal whose degree of difficulty in achievement is equal to or less than the predetermined degree is displayed. Accordingly, care can be implemented in such a manner as to near the fulfillment of the desire of the care target person.

The configuration 1 can also be similarly applied to a case where an issue or action item is recommended.

### [Configuration 2]

This configuration is a configuration that recommends an action item in accordance with the achievement rate or fulfillment rate. An action item information table where an action item (an item that a care practitioner carries out for a care target person) necessary to achieve a goal is registered, associated with the achievement rate or fulfillment rate, is stored in the storage device 42. Examples of the action item include “supervise bathing”, “assist with transfer into the tub”, and “support the body in the tub”. An action item in accordance with the achievement rate inputted in step S120, or the fulfillment rate inputted in step S122, is displayed on the display device 44 on the basis of the action item information table. The action item can be recorded, included in, for example, an update.

Consequently, an action item in accordance with the achievement rate or fulfillment rate is displayed. Accordingly, it is possible to implement care in such a manner as to near the fulfillment of the desire of the care target person.

### [Configuration 3]

This configuration is a configuration that recommends a goal on the basis of a past care track record of a care target person. A care track record information table where a care track record of a care target person is registered is stored in the storage device 42. In the example of FIG. 4, the goal information table 420 further includes a field where a care track record or identification information thereof is registered. A care track record of a care target person A is read from the care track record information table. One or more goals corresponding to a desire of the care target person A inputted in step S104 and the care track record read out are read from the goal information table 420. The goals read out are displayed on the display device 44 as goals recommended to the care target person A. For example, if a goal Y is recommended as the next goal to the care target person A who carried out an action item X in the past, the action item X is registered as a care track record in the care track record information table. The goal Y of the goal information table 420 is registered, associated with the action item X.

Consequently, the goal recommended to the care target person is displayed in accordance with the past care track record of the care target person. Accordingly, it is possible to create a care plan that is effective in fulfilling the desire of the care target person.

The configuration 3 can also be similarly applied to a case where an issue or action item is recommended.

### [Configuration 4]

This configuration is a configuration that recommends a goal on the basis of a past care track record of another care target person. A care track record information table where a care track record of a care target person is registered, associated with an attribute of the care target person, is stored in the storage device 42. In the example of FIG. 4, the goal information table 420 further includes a field where a care track record or identification information thereof is registered. A care track record corresponding to an attribute having the same or a predetermined relationship with a care target person A is read from the care track record information table. One or more goals corresponding to a desire of the care target person A inputted in step S104 and to the care track record read out are read from the goal information table 420. The goals read out are displayed on the display device 44 as goals recommended to the care target person A. For example, if a goal Y is recommended as the next goal to a care target person B of an attribute having the same or predetermined relationship with the care target person A who carried out an action item X in the past, the action item X is registered as a care track record in the care track record information table. The goal Y of the goal information table 420 is registered, associated with the action item X.

Consequently, the goal recommended to the care target person A is displayed in accordance with the past care track record of the care target person B of the attribute having the same or predetermined relationship with the care target person A. Accordingly, it is possible to create a care plan that is effective in fulfilling the desire of the care target person A.

The configuration 4 can also be similarly applied to a case where an issue or action item is recommended.

### [Configuration 5]

This configuration is a configuration that recommends a goal on the basis of a track record of past care implemented by another care practitioner. A care track record information table where a track record of care implemented by a care practitioner for a care target person is registered, associated with an attribute of the care practitioner, is stored in the storage device 42. In the example of FIG. 4, the goal information table 420 further includes a field where a care track record or identification information thereof is registered. A care track record corresponding to an attribute having the same or a predetermined relationship with a care practitioner A is read from the care track record information table. One or more goals corresponding to a desire of a care target person for whom care is implemented by the care practitioner A, the desire being inputted in step S104, and the care track record read out are read from the goal information table 420. The goals read out are displayed on the display device 44 as goals recommended to the care practitioner A. For example, if a goal Y is recommended as the next goal to a care practitioner B of an attribute having the same or predetermined relationship with the care practitioner A who carried out an action item X in the past, the action item X is registered as a care track record in the care track record information table. The action item X is registered, associated with the goal Y of the goal information table 420.

Consequently, the goal recommended to the care target person for whom the care practitioner A implements care is displayed in accordance with the track record of the past care implemented by the care practitioner B of the attribute having the same or predetermined relationship with the care practitioner A. Accordingly, it is possible to create a care plan that is effective in fulfilling a desire of the care target person.

The configuration 5 can also be similarly applied to a case where an issue or action item is recommended.

### [Configuration 6]

This configuration is a configuration that recommends a goal in accordance with a difference between the achievement rate and the fulfillment rate. If the difference between the achievement rate inputted in step S120 and the fulfillment rate inputted in step S122, both of which are related to a care target person A, or a rank of the difference is equal to or less than, or equal to or greater than, a predetermined value, a goal recommended to the care target person A among one or more goals for fulfilling the desire of the care target person A inputted in step S104 is displayed on the display device 44 on the basis of the goal information table 420. For example, one or more goals for fulfilling a desire of a care target person is registered, associated with a difference between the achievement rate and the fulfillment rate, or a rank thereof, in the goal information table 420. The difference between the achievement rate inputted in step S120 and the fulfillment rate inputted in step S122, both of which are related to the care target person A, or the rank thereof is calculated. If the calculated difference or rank is equal to or less than, or equal to or greater than, the predetermined value, one or more goals corresponding to the calculated difference or rank are read from the goal information table 420. The goals read out are displayed on the display device 44 as goals recommended to the care target person A.

Consequently, if the difference between the achievement rate and the fulfillment rate, or the rank thereof is equal to less than, equal to or greater than, the predetermined value, a goal recommended to the care target person is displayed. Accordingly, it is possible to implement care in such a manner as to near the fulfillment of the desire of the care target person.

The configuration 6 can also be similarly applied to a case where an issue or action item is recommended.

### [Configuration 7]

This configuration is a configuration that recommends a goal if the difference between the achievement rate and the fulfillment rate is large in the configuration 6. One or more goals for fulfilling the desire of the care target person, and the degrees of difficulty in achieving the one or more goals are registered, associated with the desire, in the goal information table 420. In the example of FIG. 4, the goal information table 420 further includes a field where the degree of difficulty in achieving a goal is registered. If the difference between the achievement rate and the fulfillment rate is equal to or greater than the predetermined value, a goal having a different degree of difficulty in achievement from a previous input is displayed on the display device 44. For example, a goal having a lower degree of difficulty in achievement than the previous input is displayed.

Consequently, if the difference between the achievement rate and the fulfillment rate is equal to or greater than the predetermined value, a goal having a different degree of difficulty in achievement from the previous input is displayed. Accordingly, it is possible to implement care in such a manner as to near the fulfillment of the desire of the care target person.

The configuration 7 can also be similarly applied to a case where an issue or action item is recommended.

### [Configuration 8]

This configuration is a configuration that determines whether or not the setting of a goal is appropriate, from a past track record and inputted information. It is determined whether or not the goal inputted in step S110 can be achieved on the basis of care management information including a goal, an issue, and a target achievement date, which were inputted in the past, and the goal, the issue, and the target achievement date, which were inputted in steps S110, S114, and S116. For example, care management information including a goal, an issue, and a target achievement date, which were inputted in the past and include the same combination of a goal and an issue as the goal and the issue, which were inputted in steps S110 and S114, is read from the storage device 42. If the target achievement date inputted in step S116 is earlier than the target achievement date included in the care management information read out, it is determined that the goal inputted in step S110 cannot be achieved.

Consequently, it is determined whether or not the goal can be achieved, from the past track record and the inputted information. Accordingly, it is possible to prevent the creation of a care plan with a low possibility of fulfilling the desire of the care target person.

### [Configuration 9]

This configuration is a configuration that displays candidates for a target achievement date. Candidates for a target achievement date scheduled to achieve an inputted goal are displayed on the basis of a target achievement date information table where one or more target achievement dates scheduled to achieve a goal of the goal information table 420 are registered, associated with the goal. Any of the displayed target achievement dates is inputted. Care management information including the inputted target achievement date is registered in the storage device 42. The target achievement date is displayed per care target person on the basis of the care management information of the storage device 42.

Consequently, one or more target achievement dates scheduled to achieve the selected goal are displayed, and the care practitioner is simply required to select any of the target achievement dates. Accordingly, even a care practitioner with little experience can create a care plan for fulfilling the desire of the care target person compared to before.

### [Configuration 10]

This configuration is a configuration that displays candidates for an achievement rate. Candidates for an achievement rate of achievement of an inputted goal are displayed on the basis of an achievement rate information table where one or more achievement rates of a goal of the goal information table 420 are registered, associated with the goal. Any of the displayed achievement rates is inputted. Care management information including the inputted achievement rate is registered in the storage device 42. The achievement rate is displayed per care target person on the basis of the care management information of the storage device 42.

Consequently, one or more achievement rates of the selected goal are displayed, and the care practitioner is simply required to select any of the achievement rates. Accordingly, even a care practitioner with little experience can create a care plan for fulfilling the desire of the care target person compared to before.

### [Configuration 11]

This configuration is a configuration that displays candidates for a fulfillment rate. Candidates for a fulfillment rate obtained by achieving an inputted goal are displayed on the basis of a fulfillment rate information table where one or more fulfillment rates of a desire that is attempted to be fulfilled by achieving a goal of the goal information table 420 are registered, associated with the goal. Any of the displayed fulfillment rates is inputted. Care management information including the inputted fulfillment rate is registered in the storage device 42. The fulfillment rate is displayed per care target person on the basis of the care management information of the storage device 42.

Consequently, one or more fulfillment rates of the desire that is attempted to be fulfilled by achieving the selected goal are displayed, and the care practitioner is simply required to select any of the fulfillment rates. Accordingly, even a care practitioner with little experience can create a care plan for fulfilling the desire of the care target person compared to before.

### [Configuration 12]

This configuration is a configuration that creates a desire fulfillment history. For example, if the fulfillment rate reaches 100%, a desire fulfillment history including the date when the desire came true is created. Care management information including the created desire fulfillment history is registered in the storage device 42. Moreover, at this point in time, it can also be configured in such a manner as to prompt to set a new goal.

### [Configuration 13]

This configuration is a configuration that creates a goal achievement history. For example, if the achievement rate reaches 100%, a goal achievement history including the date when the goal was achieved is created. Care management information including the created goal achievement history is registered in the storage device 42. Moreover, at this point in time, it can also be configured in such a manner as to prompt to set a new goal.

Moreover, in terms of the configurations 2 to 7, it can also be configured in such a manner that a recommendation is made by AI (Artificial Intelligence).

Moreover, in the above first embodiment and the modifications thereof, it is configured in such a manner that goal information indicating one or more goals for fulfilling a desire of a care target person is registered, associated with the desire, in the goal information table 420, and candidates for a goal for fulfilling the desire of the care target person are displayed on the basis of the goal information table 420. However, the configuration is not limited to this. It can also be configured in such a manner that goal information indicating one or more goals for fulfilling a desire of a family of a care target person is registered, associated with the desire, in the goal information table 420, and candidates for a goal for fulfilling the desire of the family of the care target person are displayed on the basis of the goal information table 420.

Moreover, in the above first embodiment and the modifications thereof, the care support apparatus 100 being a single apparatus is realized. However, not limited to the care support apparatus 100, but also a network system can be realized. A care support server and a plurality of user terminals are connected to a network such as the Internet. The care support server is configured including functions equivalent to the care support apparatus 100. The point of receiving information from the user terminal instead of input through the input device 40, and the point of transmitting information to the user terminal instead of display on the display device 44 are different from the care support apparatus 100.

Moreover, in the above first embodiment and the modifications thereof, it is configured in such a manner as to use the storage device 42 included in the care support apparatus 100. However, the configuration is not limited to this. It can also be configured in such a manner as to use an external storage device such as a database server.

Moreover, in the above first embodiment and the modifications thereof, a description is given of the case where the program stored in advance in the ROM 32 is executed when the process illustrated in the flowchart of FIG. 6 is executed. However, the configuration is not limited to this. A program including the procedure may be read into the RAM 34 from a storage medium where the program is stored, and executed.

The storage medium is a semiconductor storage medium such as RAM or ROM, a magnetic storage-type storage medium such as an FD or HD, an optical reading-type storage medium such as a CD, CDV, LD, or DVD, or a magnetic storage-type/optical reading-type storage medium such as an MO, and includes every storage medium regardless of a reading method such as electronic, magnetic, and optical reading as long as it is a computer-readable storage medium.

Moreover, the present invention is not limited to the above first embodiment and the modifications thereof, and can also be applied to other cases within the scope that does not depart from the gist of the present invention. For example, the present invention can be applied to cases of supporting a care practitioner in implementing care (for example, taking care, protection, caregiving, and nursing) for a care target person not limited to in the caregiving field but also in the medical (for example, rehab or treatment) or (for example, physical, intellectual, or mental) welfare field. In this case, if a database is constructed for items of the relevant field, it can be linked to a common format.

### Second Embodiment

Next, a second embodiment of the present invention is described. FIGS. 1 and 10 to 19 are diagrams illustrating the embodiment.

Firstly, the configuration of the embodiment is described.

As illustrated in FIG. 1, a care support apparatus 100 includes a CPU 30 that performs computations and controls the entire system on the basis of a control program, a ROM 32 that stores in advance, for example, the control program of the CPU 30 in a predetermined area, a RAM 34 for storing data read from, for example, the ROM 32, and computation results necessary in the course of computation of the CPU 30, and an I/F 38 through which data is inputted into and outputted from an external device. They are connected to each other with a bus 39 being a signal line for transferring data in such a manner as to be able to exchange data.

The I/F 38 is connected to, as external devices, an input device 40 including a keyboard and a mouse, which can input data as human interfaces, a storage device 42 that stores, for example, data and tables as files, and a display device 44 that displays screens on the basis of image signals.

Next, the data structure of the storage device 42 is described.

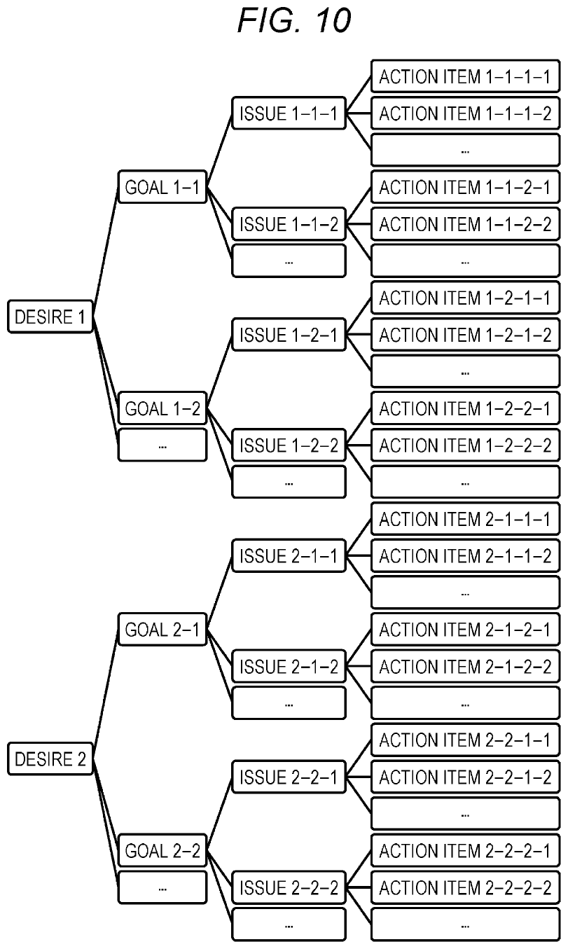

FIG. 10 is a conceptual diagram illustrating the data coupling relationship of tables 400 to 460.

Care practitioners, mainly care practitioners with rich experience, list in advance (1) candidates for a desire of a care target person, (2) candidates for a goal for fulfilling the desire, (3) candidates for an issue (a problem or a point to be improved) in achieving the goal, and (4) candidates for an action item (an item that a care practitioner carries out on the care target person) necessary to achieve the goal.

As illustrated in FIG. 10, for one desire, one or more goals for fulfilling the desire can be set. In the example of FIG. 10, a goal 1-1, a goal 1-2, and other goals are set for a desire 1. A goal 2-1, a goal 2-2, and other goals are set for a desire 2. Each desire is associated with goals to be set. Accordingly, the tendency and the content may be common or similar in the goals 1-1, 1-2, 2-1, and 2-2. However, the goals 1-1, 1-2, 2-1, and 2-2 are basically independent of each other.

Next, for one goal, one or more issues in achieving the goal can be set. In the example of FIG. 10, an issue 1-1-1, an issue 1-1-2, and other issues are set for the goal 1-1. An issue 1-2-1, an issue 1-2-2, and other issues are set for the goal 1-2. An issue 2-1-1, an issue 2-1-2, and other issues are set for the goal 2-1. An issue 2-2-1, an issue 2-2-2, and other issues are set for the goal 2-2. Each goal is associated with issues to be set. Accordingly, the tendency and the content may be common or similar in the issues 1-1-1, 1-1-2, 1-2-1, 1-2-2, 2-1-1, 2-1-2, 2-2-1, and 2-2-2. However, the issues 1-1-1, 1-1-2, 1-2-1, 1-2-2, 2-1-1, 2-1-2, 2-2-1, and 2-2-2 are basically independent of each other.

Next, for one issue, one or more action items necessary to solve the issue can be set. In the example of FIG. 10, an action item 1-1-1-1, an action item 1-1-1-2, and other action items are set for the issue 1-1-1. An action item 1-1-2-1, an action item 1-1-2-2, and other action items are set for the issue 1-1-2. An action item 1-2-1-1, an action item 1-2-1-2, and other action items are set for the issue 1-2-1. An action item 1-2-2-1, an action item 1-2-2-2, and other action items are set for the issue 1-2-2. Moreover, an action item 2-1-1-1, an action item 2-1-1-2, and other action items are set for the issue 2-1-1. An action item 2-1-2-1, and an action item 2-1-2-2, and other items are set for the issue 2-1-2. An action item 2-2-1-1, an action item 2-2-1-2, and other action items are set for the issue 2-2-1. An action item 2-2-2-1, an action item 2-2-2-2, and other action items are set for the issue 2-2-2. Each issue is associated with action items to be set. Accordingly, the tendency and the content may be common or similar in the action items 1-1-1-1, 1-1-1-2, 1-1-2-1, 1-1-2-2, 1-2-1-1, 1-2-1-2, 1-2-2-1, 1-2-2-2, 2-1-1-1, 2-1-1-2, 2-1-2-1, 2-1-2-2, 2-2-1-1, 2-2-1-2, 2-2-2-1, and 2-2-2-2. However, the action items 1-1-1-1, 1-1-1-2, 1-1-2-1, 1-1-2-2, 1-2-1-1, 1-2-1-2, 1-2-2-1, 1-2-2-2, 2-1-1-1, 2-1-1-2, 2-1-2-1, 2-1-2-2, 2-2-1-1, 2-2-1-2, 2-2-2-1, and 2-2-2-2 are basically independent of each other.

This data structure is realized by the desire information table 400, the goal information table 420, the issue information table 440, and the action item information table 460.

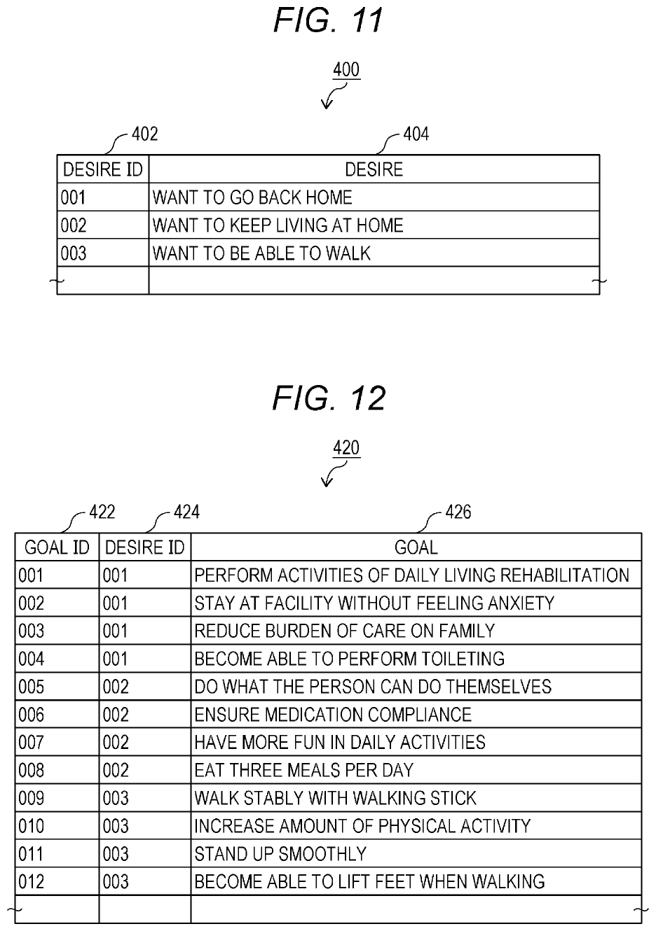

FIG. 11 is a diagram illustrating the data structure of the desire information table 400.

As illustrated in FIG. 11, the desire information table 400 is stored in the storage device 42.

One record is registered per desire in the desire information table 400. Each record is configured including a field 402 where a desire ID for identifying a desire is registered, and a field 404 where the desire (in text) is registered. In the example of FIG. 11, “001” is registered as the desire ID, and “want to go back home” as the desire in a record in the first row. This indicates that the desire identified by the desire ID “001” is “want to go back home.”

FIG. 12 is a diagram illustrating the data structure of the goal information table 420.

As illustrated in FIG. 12, the goal information table 420 is stored in the storage device 42.

One record is registered per goal in the goal information table 420. Each record is configured including a field 422 where a goal ID for identifying a goal is registered, a field 424 where a desire ID of a desire attempted to be fulfilled by achieving the goal is registered, and a field 426 where the goal (in text) is registered. In the example of FIG. 12, “001” is registered as the goal ID, “001” as the desire ID, and “perform activities of daily living rehabilitation” as the goal in a record in the first row. This indicates that the goal identified by the goal ID “001” is “perform activities of daily living rehabilitation” and is set as the goal for fulfilling the desire “want go back home” of the desire ID “001”.

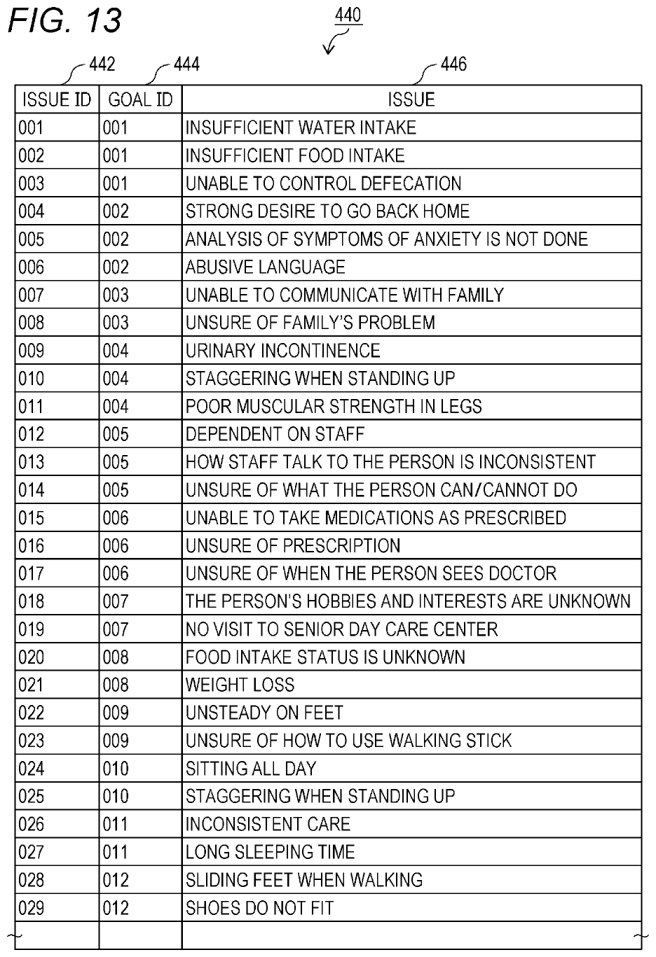

FIG. 13 is a diagram illustrating the data structure of the issue information table 440.

As illustrated in FIG. 13, the issue information table 440 is stored in the storage device 42.

One record is registered per issue in the issue information table 440. Each record is configured including a field 442 where an issue ID for identifying an issue is registered, a field 444 where a goal ID of a goal attempted to be achieved by solving the issue is registered, and a field 446 where the issue (in text) is registered. In the example of FIG. 13, “001” is registered as the issue ID, and “001” as the goal ID, and “insufficient water intake” as the issue in a record in the first row. This indicates that the issue identified by the issue ID “001” is “insufficient water intake” and is set as the issue in achieving the goal “perform activities of daily living rehabilitation” of the goal ID “001”.

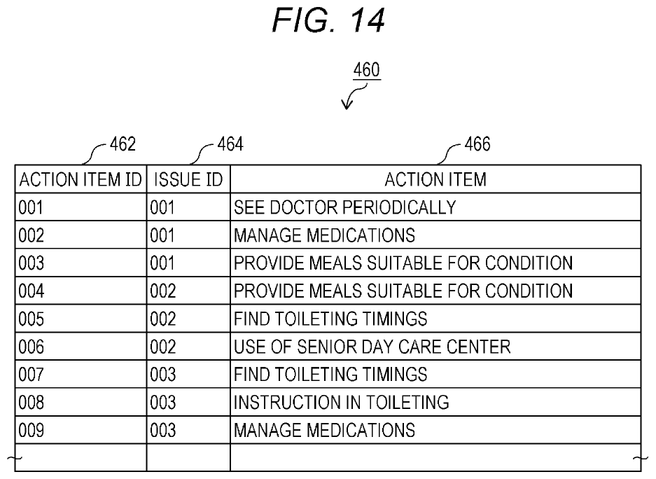

FIG. 14 is a diagram illustrating the data structure of the action item information table 460.

As illustrated in FIG. 14, the action item information table 460 is stored in the storage device 42.

One record is registered per action item in the action item information table 460. Each record is configured including a field 462 where an action item ID for identifying an action item is registered, a field 464 where an issue ID of an issue attempted to be solved by the action item is registered, and a field 466 where the action item (in text) is registered. In the example of FIG. 14, “001” is registered as the action item ID, “001” as the issue ID, “see a doctor periodically” as the action item in a record in the first row. This indicates that the action item identified by the action item ID “001” is “see a doctor periodically” and is set as the action item necessary to solve the issue “insufficient water intake” of the issue ID “001”. The issue is associated with the goal. Accordingly, the action item is also associated indirectly with the goal or desire.

Next, the operation of the embodiment is described.

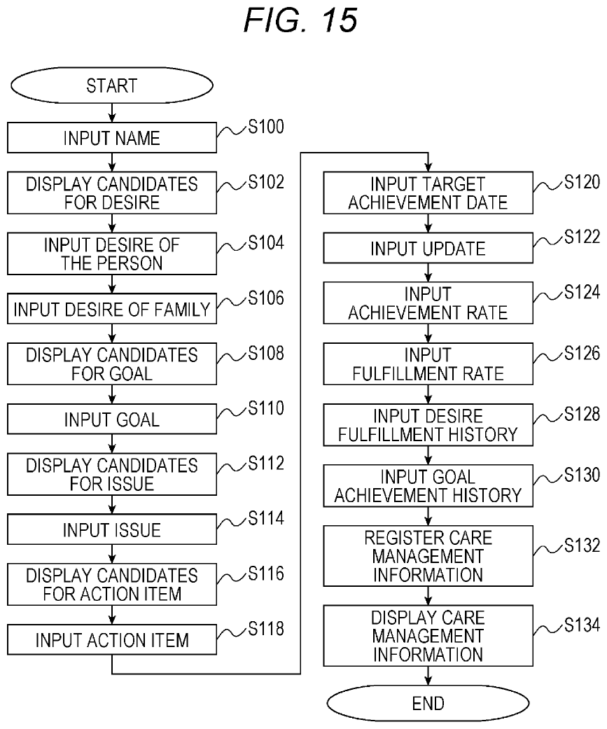

The CPU 30 includes, for example, an MPU. The CPU 30 starts a predetermined program stored in a predetermined area of the ROM 32, and executes a care support process illustrated in a flowchart of FIG. 15 in accordance with the program.

FIG. 15 is a flowchart illustrating the care support process.

The care support process is a process that is started at the request of a user such as a care practitioner. When the CPU 30 executes the care support process, the procedure moves on to step S100 first as illustrated in FIG. 15.

In step S100, if the input of the name of a care target person is requested, the name of the care target person is inputted through the input device 40.

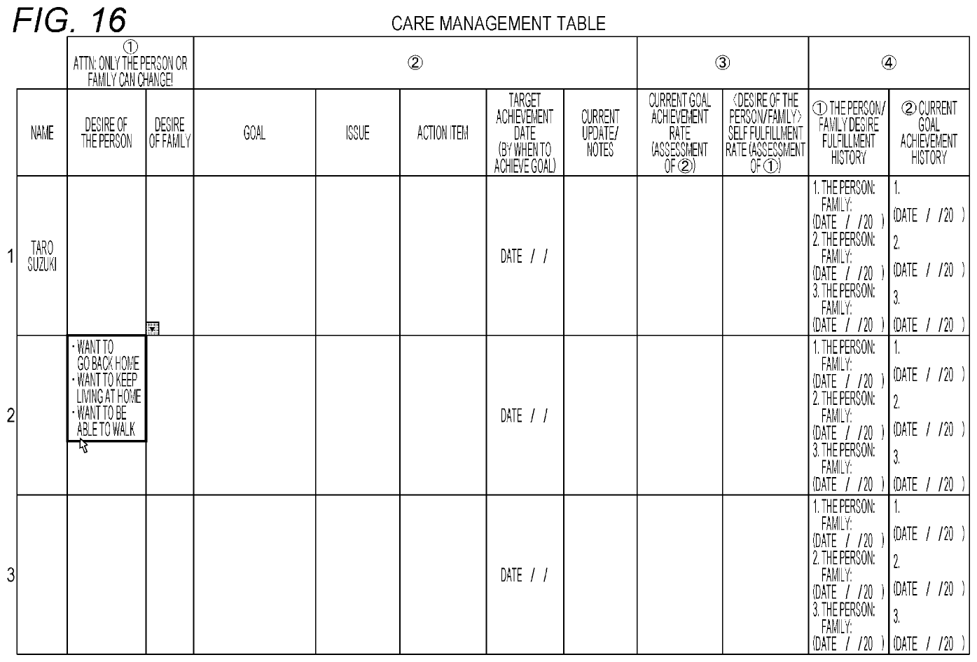

FIG. 16 is a screen illustrating a care management table in a case of inputting a desire from a plurality of candidates.

Next, the procedure moves on to step S102. If the input of a desire of the care target person is requested, one or more desires are read from the desire information table 400. As illustrated in FIG. 16, candidates for the desire of the care target person are displayed on the display device 44. A care practitioner selects any of the displayed candidates. In the example of FIG. 16, “want to go back home”, “want to keep living at home”, “want to be able to walk” are displayed as the candidates for the desire. It is assumed that the care practitioner selects, for example, “want to go back home”. If the desire is selected, the procedure moves on to step S104. The selected desire is inputted through the input device 40.

Next, the procedure moves on to step S106. If the input of a desire of a family of the care target person is requested, the desire of the family of the care target person is inputted through the input device 40.

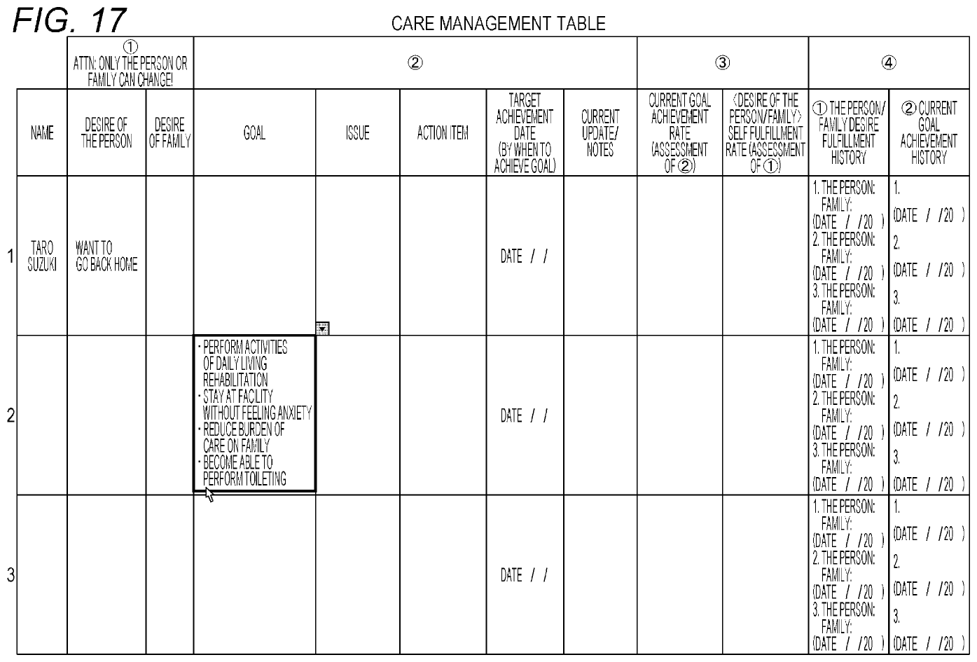

FIG. 17 is a screen illustrating a care management table in a case of inputting a goal from a plurality of candidates.

Next, the procedure moves on to step S108. If the input of a goal is requested, one or more goals corresponding to a desire ID of the desire inputted in step S104 are read from the goal information table 420. As illustrated in FIG. 17, candidates for a goal for fulfilling the desire are displayed on the display device 44. The care practitioner selects any of the displayed candidates. In the example of FIG. 17, “perform activities of daily living rehabilitation”, “stay at a facility without feeling anxiety”, “reduce the burden of care on the family”, and “become able to perform toileting” are displayed as the candidates for the goal. It is assumed that the care practitioner selects, for example, “perform activities of daily living rehabilitation”. If the goal is selected, the procedure moves on to step S110. The selected goal is inputted through the input device 40.

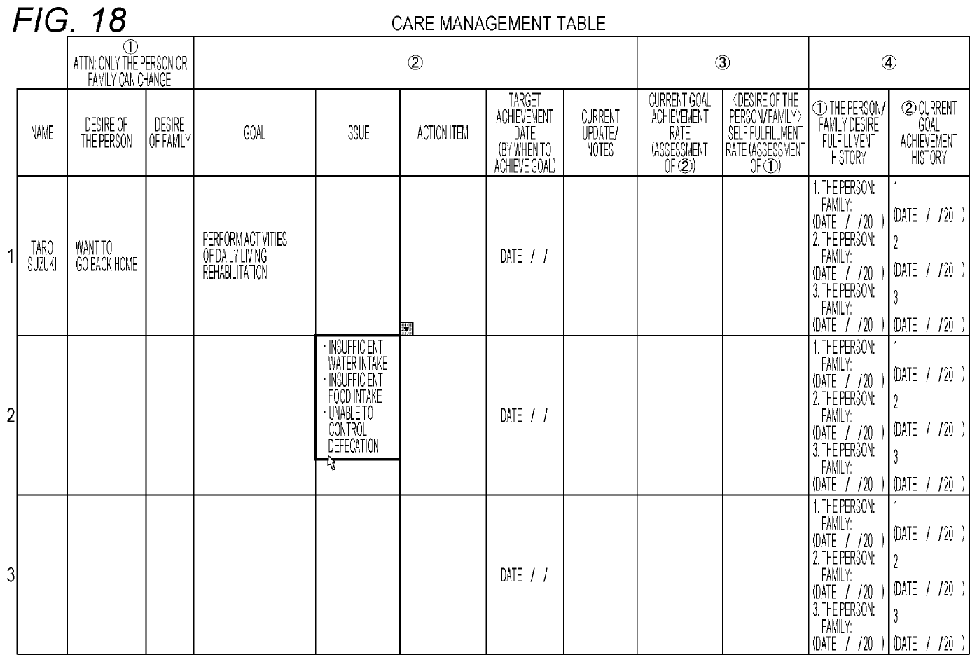

FIG. 18 is a screen illustrating a care management table in a case of inputting an issue from a plurality of candidates.

Next, the procedure moves on to step S112. If the input of an issue is requested, one or more issues corresponding to a goal ID of the goal inputted in step S110 are read from the issue information table 440. As illustrated in FIG. 18, candidates for an issue in achieving the goal are displayed on the display device 44. The care practitioner selects any of the displayed candidates. In the example of FIG. 18, “insufficient water intake”, “insufficient food intake”, and “unable to control defecation” are displayed as the candidates for the issue. It is assumed that the care practitioner selects, for example, “insufficient water intake”. If the issue is selected, the procedure moves on to step S114. The selected issue is inputted through the input device 40.

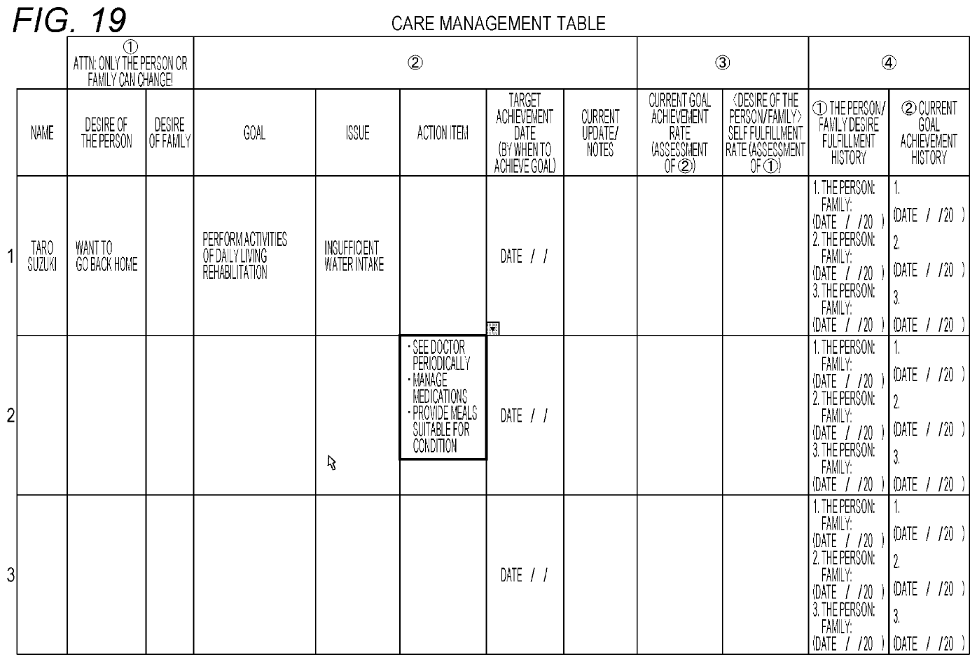

FIG. 19 is a screen illustrating a care management table in a case of inputting an action item from a plurality of candidates.

Next, the procedure moves on to step S116. If the input of an action item is requested, one or more action items corresponding to an issue ID of the issue inputted in step S114 are read from the action item information table 460. As illustrated in FIG. 19, candidates for an action item necessary to solve the issue are displayed on the display device 44. The care practitioner selects any of the displayed candidates. In the example of FIG. 19, “see a doctor periodically”, “manage medications”, “provide meals suitable for the condition” are displayed as the candidates for the action item. It is assumed that the care practitioner selects, for example, “see a doctor periodically”. If the action item is selected, the procedure moves on to step S118. The selected action item is inputted through the input device 40.

Next, the procedure moves on to S120. If the input of a target achievement date is requested, a target achievement date scheduled to achieve the goal is inputted through the input device 40.

Next, the procedure moves on to step S122. If the input of an update is requested, an update of a previous input is inputted through the input device 40.

Next, the procedure moves on to step S124. If the input of an achievement rate is requested, the achievement rate of the goal (in percentage) is inputted through the input device 40.

Next, the procedure moves on to step S126. If the input of a fulfillment rate is requested, the fulfillment rate of the desire (in percentage) is inputted through the input device 40. The fulfillment rate is inputted on the basis of an answer from the care target person or family.

Next, the procedure moves on to step S128. If the input of a desire fulfillment history is requested, a desire fulfillment history including the date when the desire came true is inputted through the input device 40. The desire fulfillment history is inputted on the basis of an answer form the care target person or family.

Next, the procedure moves on to step S130. If the input of a goal achievement history is requested, a goal achievement history including the date when the goal was achieved is inputted through the input device 40.

Next, the procedure moves on to step S132. Care management information including the name, the desire of the care target person, the desire of the family, the goal, the issue, the action item, the target achievement date, the update, the achievement rate, the fulfillment rate, the desire fulfillment history, and the goal achievement history, which were inputted in steps S100, S104, S106, S110, S114, S118, and S120 to S130, is registered in the storage device 42. The care management information is registered per care target person.

The procedure then moves on to step S134. The name, the desire of the care target person, the desire of the family, the goal, the issue, the action item, the target achievement date, the update, the achievement rate, the fulfillment rate, the desire fulfillment history, and the goal achievement history are displayed per care target person on the display device 44 on the basis of the care management information of the storage device 42. For example, items corresponding to the care management information are displayed in the fields of the care management table of FIGS. 16 to 19, respectively.

Next, the effects of the embodiment are described.

In the embodiment, a desire of a care target person is inputted. Candidates for a goal for fulfilling the inputted desire are displayed on the basis of the goal information table 420. Any of the displayed goals is inputted. One or more issues in achieving the inputted goal are displayed on the basis of the issue information table 440. Any of the displayed issues is inputted. Care management information including the name of the care target person, and the inputted desire, goal, and issue is registered in the storage device 42. The name, the desire, the goal, and the issue are displayed per care target person on the basis of the care management information of the storage device 42.

Consequently, one or more goals for fulfilling the desire of the care target person are displayed, and a care practitioner is simply required to select any of the goals, and furthermore one or more issues in achieving the selected goal are displayed, and the care practitioner is simply required to select any of the issues. Accordingly, even if a care practitioner with little experience can create a care plan for fulfilling the desire of the care target person compared to before. Moreover, it is possible to grasp the name, the desire, the goal, and the issue per care target person. Accordingly, it is possible to implement care for fulfilling the desire of the care target person.

Furthermore, in the embodiment, one or more action items necessary to solve the inputted issue are displayed on the basis of the action item information table 460. Any of the displayed action items is inputted. Care management information including the name of the care target person, and the inputted desire, goal, issue, and action item is registered in the storage device 42. The name, the desire, the goal, the issue, and the action item are displayed per care target person on the basis of the care management information of the storage device 42.

Consequently, it is possible to grasp the name, the desire, the goal, the issue, and the action item per care target person. Accordingly, it is possible to implement care for fulfilling the desire of the care target person.

Furthermore, in the embodiment, an achievement rate of the inputted goal is inputted. A fulfillment rate of the inputted desire is inputted. Care management information including the name of the care target person, and the inputted desire, goal, issue, action item, achievement rate, and fulfillment rate is registered in the storage device 42. The name, the desire, the goal, the issue, the action item, the achievement rate, and the fulfillment rate are displayed per care target person on the basis of the care management information of the storage device 42.

Consequently, it is possible to grasp the name, the desire, the goal, the issue, the action item, the achievement rate, and the fulfillment rate per care target person. Accordingly, it is possible to more appropriately implement care for fulfilling the desire of the care target person.

Furthermore, in the embodiment, a target achievement date scheduled to achieve the inputted goal is inputted. An update of a previous input is inputted. A desire fulfillment history including the date when the inputted desire came true is inputted. A goal achievement history including the date when the inputted goal was achieved is inputted. Care management information including the name of the care target person, and the inputted desire, goal, issue, action item, achievement rate, fulfillment rate, target achievement date, update, desire fulfillment history, and goal achievement history is registered in the storage device 42. The name, the desire, the goal, the issue, the action item, the achievement rate, the fulfillment rate, the target achievement date, the update, the desire fulfillment history, and the goal achievement history are displayed per care target person on the basis of the care management information of the storage device 42.

Consequently, it is possible to grasp the name, the desire, the goal, the issue, the action item, the achievement rate, the fulfillment rate, the target achievement date, the update, the desire fulfillment history, and the goal achievement history per care target person. Accordingly, it is possible to more appropriately implement care for fulfilling the desire of the care target person.

In the embodiment, the storage device 42 corresponds to the goal information storage means of aspect B1 or B21, the issue information storage means of aspect B2, the action item information storage means of aspect B1 or B21, or the care management information storage means of any of aspects B1 to B4 and B21. Step S100 corresponds to the identification information input means of any of aspects B1 to B4 and B21, or the identification information input step of aspect B21. Moreover, step S104 corresponds to the desire input means of any of aspects B1 to B4 and B21, or the desire input step of aspect B21. Step S108 corresponds to the goal presenting means of aspect B1 or B21, or the goal presenting step of aspect B21. Step S110 corresponds to the goal input means of any of aspects B1 to B4 and B21, or the goal input step of aspect B21.

Moreover, in the embodiment, step S112 corresponds to the issue presenting means of aspect B2. Step S114 corresponds to the issue input means of aspect B2. Step S116 corresponds to the action item presenting means of aspect B1 or B21, or the action item presenting step of aspect B21. Moreover, step S118 corresponds to the action item input means of any of aspects B1 to B4 and B21, or the action item input step of aspect B21. Step S120 corresponds to the target achievement date input means of aspect B4. Step S122 corresponds to the update input means of aspect B4.

Moreover, in the embodiment, step S124 corresponds to the achievement degree input means of aspect B3 or B4. Step S126 corresponds to the fulfillment degree input means of aspect B3 or B4. Step S128 corresponds to the actual fulfillment date input means of aspect B4. Step S130 corresponds to the actual achievement date input means of aspect B4. Moreover, step S132 corresponds to the care management information registration means of any of aspects B1 to B4 and B21, or the care management information registration step of aspect B21. Step S134 corresponds to the care management information presenting means of any of aspects B1 to B4 and B21, or the care management information presenting step of aspect B21.

### Third Embodiment

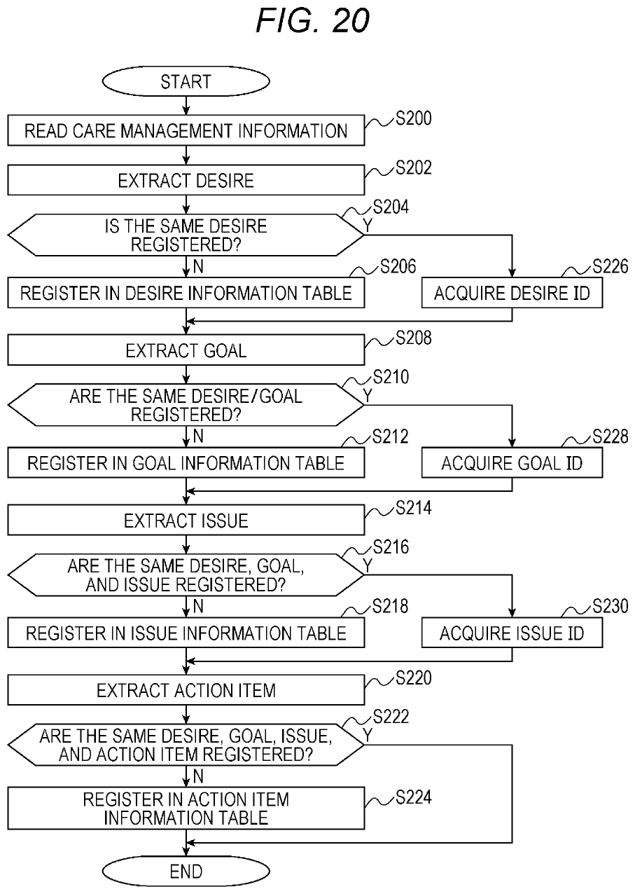

Next, a third embodiment of the present invention is described. FIG. 20 is a diagram illustrating the embodiment. Only features different from the above second embodiment are described below. The same reference signs are assigned to overlapping features, and descriptions thereof are omitted.

The embodiment presents a process of registering information in the tables 400 to 460 in the above second embodiment on the basis of the care management information where the desire, the goal, the issue, and the action item are associated.

Firstly, the configuration of the embodiment is described.

One or more pieces of the care management information are stored in the storage device 42. In the care management information, (1) a desire of a care target person or his/her family, (2) a goal for fulfilling the desire, (3) an issue in achieving the goal, and (4) an action item necessary to solve the issue are associated. Moreover, the care management information may be information on, for example, a desire inputted and registered by the care support apparatus 100 according to the above second embodiment, information on, for example, a desire manually inputted and registered by, for example, the care practitioner, information obtained by performing a character recognition process such as OCR (Optical Character Recognition) on a document filled by, for example, the care practitioner, or information obtained by performing an imaging process on a document filled by, for example, the care practitioner.

Next, the operation of the embodiment is described.

The CPU 30 starts a predetermined program stored in a predetermined area of the ROM 32, and executes a care support information registration process illustrated in a flowchart of FIG. 20 in accordance with the program.

FIG. 20 is a flowchart illustrating the care support information registration process.

The care support information registration process is a process that is started at the request of a user such as a care practitioner. If the CPU 30 executes the care support information registration process, the procedure moves on to step S200 first as illustrated in FIG. 20.

In step S200, care management information specified by the user is read from the storage device 42. The procedure moves on to step S202.

In step S202, a desire is extracted from the care management information read in step S200. If the care management information is image information (if not in text format), a desire is extracted after the character recognition process such as OCR is performed on the care management information. The same shall apply below in steps S208, S214, and S220.

Next, the procedure moves on to step S204. It is determined whether or not the same desire as the desire extracted in step S202 is registered in the desire information table 400. If it is determined that the same desire is not registered (NO), the procedure moves on to step S206. A new desire ID is issued. The desire extracted in step S204 is registered, associated with the issued desire ID, in the desire information table 400. The procedure moves on to step S208.

In step S208, a goal is extracted from the care management information read out in step S200. The procedure moves on to step S210. It is determined whether or not the same desire as the desire extracted in step S202 is registered in the desire information table 400, and whether or not the same goal as the goal extracted in step S208 is registered in the goal information table 420. As a result, if it is determined that the same desire and the same goal are not registered (NO), the procedure moves on to step S212. A new goal ID is issued. The goal extracted in step S208 is registered, associated with the issued goal ID and with the issued or acquired desire ID, in the goal information table 420. The procedure moves on to step S214.

In step S214, an issue is extracted from the care management information read in step S200. The procedure moves on to step S216. It is determined whether or not the same desire as the desire extracted in step S202 is registered in the desire information table 400, whether or not the same goal as the goal extracted in step S208 is registered in the goal information table 420, and whether or not the same issue as the issue extracted in step S214 is registered in the issue information table 440. As a result, if it is determined that the same desire, the same goal, and the same issue are not registered (NO), the procedure moves on to step S218. A new issue ID is issued. The issue extracted in step S214 is registered, associated with the issued issue ID and the issued or acquired goal ID, in the issue information table 440. The procedure moves on to step S220.

In step S220, an action item is extracted from the care management information read in step S200. The procedure moves on to step S222. It is determined whether or not the same desire as the desire extracted in step S202 is registered in the desire information table 400, whether or not the same goal as the goal extracted in step S208 is registered in the goal information table 420, whether or not the same issue as the issue extracted in step S214 is registered in the issue information table 440, and whether or not the same action item as the action item extracted in step S220 is registered in the action item information table 460. As a result, if it is determined that the same desire, the same goal, the same issue, and the same action item are not registered (NO), the procedure moves on to step S224. A new action item ID is issued. The action item extracted in step S220 is registered, associated with the issued action item ID and the issued or acquired issue ID, in the action item information table 460. A series of steps ends.

On the other hand, if in step S204 it is determined that the same desire is registered (YES), the procedure moves on to step S226. A desire ID corresponding to the desire extracted in step S202 is acquired from the desire information table 400. The procedure moves on to step S208.

On the other hand, if in step S210 it is determined that the same desire and the same goal are registered (YES), the procedure moves on to step S228. A goal ID corresponding to the issued or acquired desire ID and to the goal extracted in step S208 is acquired from the goal information table 420. The procedure moves on to step S214.

On the other hand, if in step S216 it is determined that the same desire, the same goal, and the same issue are registered (YES), the procedure moves on to step S230. An issue ID corresponding to the issued or acquired desire ID and goal ID and to the issue extracted in step S214 is acquired from the issue information table 440. The procedure moves on to step S220.

On the other hand, if in step S222 it is determined that the same desire, the same goal, the same issue, and the same action item are registered (YES), a series of steps ends.

Next, the effects of the embodiment are described.

In the embodiment, a desire, a goal, an issue, and an action item are extracted from care management information. The extracted goal is registered, associated with the extracted desire, in the goal information table 420. The extracted issue is registered, associated with the extracted goal, in the issue information table 440. The extracted action item is registered, associated with the extracted issue, in the action item information table 460.

Consequently, the goal associated with the desire, the issue associated with the goal, and the action item associated with the issue can be obtained.

In the embodiment, the storage device 42 corresponds to the goal information storage means of aspect B16, B18, B24, or B25, the issue information storage means of aspect B16 or B24, or the action item information storage means of aspect B17, B18, or B25. Step S200 corresponds to the care management information acquisition means of any of aspects B16 to B18, B24, and B25, or the care management information acquisition step of aspect B24 or B25. Moreover, step S202 corresponds to the desire extraction means of aspect B16, B18, B24, or B25, or the desire extraction step of aspect B24 or B25. Step S208 corresponds to the goal extraction means of any of aspects B16 to B18, B24, and B25, or the goal extraction step of aspect B24 or B25.

Moreover, in the embodiment, step S212 corresponds to the goal information registration means of aspect B16, B18, B24, or B25, or the goal information registration step of aspect B24 or B25. Step S214 corresponds to the issue extraction means of aspect B16 or B24, or the issue extraction step of aspect B24. Moreover, step S218 corresponds to the issue information registration means of aspect B16 or B24, or the issue information registration step of aspect B24. Step S220 corresponds to the action item extraction means of aspect B17, B18, or B25, or the action item extraction step of aspect B25.

Moreover, in the embodiment, step S224 corresponds to the action item information registration means of aspect B17, B18, or B25, or the action item information registration step of aspect B25.

### [Modifications]

The following configurations can be employed as modifications of the above second and third embodiments.

[Configuration 1] This configuration is a configuration that narrows down candidates for a goal according to the achievement rate or fulfillment rate. One or more goals for fulfilling a desire of a care target person, and the degrees of difficulty in achieving the one or more goals are registered, associated with the desire, in the goal information table 420. In the example of FIG. 12, the goal information table 420 further includes a field where the degree of difficulty in achieving a goal is registered. In step S108, if a previous achievement rate inputted in step S124 or a previous fulfillment rate inputted in step S126, both of which are related to a care target person A, is equal to or less than a predetermined value, a goal whose degree of difficulty in achievement is equal to or less than a predetermined degree among one or more goals for fulfilling the desire of the care target person A inputted in step S104 is displayed on the display device 44 on the basis of the goal information table 420.

Consequently, if the achievement rate or fulfillment rate is equal to or less than the predetermined value, a goal whose degree of difficulty in achievement is equal to or less than the predetermined degree is displayed. Accordingly, it is possible to implement care in such a manner as to near the fulfillment of the desire of the care target person.

The configuration 1 can also be similarly applied to a case where an issue or action item is recommended.

[Configuration 2] This configuration is a configuration that recommends an action item in accordance with the achievement rate or fulfillment rate. An action item information table where an action item is registered, associated with the achievement rate or fulfillment rate, is stored in the storage device 42. Examples of the action item include “supervise bathing”, “assist with transfer into the tub”, and “support the body in the tub”. An action item in accordance with the achievement rate inputted in step S124 or the fulfillment rate inputted in step S126 is displayed on the display device 44 on the basis of the action item information table. The action item can be recorded, included in, for example, an update.

Consequently, the action item in accordance with the achievement rate or fulfillment rate is displayed. Accordingly, it is possible to implement care in such a manner as to near the fulfillment of the desire of the care target person.

[Configuration 3] This configuration is a configuration that recommends a goal on the basis of a past care track record of a care target person. A care track record information table where a care track record of a care target person is registered is stored in the storage device 42. In the example of FIG. 12, the goal information table 420 further includes a field where a care track record or identification information thereof is registered. A care track record of a care target person A is read from the care track record information table. One or more goals corresponding to the desire of the care target person A inputted in step S104 and to the care track record read out are read from the goal information table 420. The goals read out are displayed on the display device 44 as goals recommended to the care target person A. For example, if a goal Y is recommended as the next goal to the care target person A who carried out an action item X in the past, the action item X is registered as a care track record in the care track record information table. The goal Y of the goal information table 420 is registered, associated with the action item X.

Consequently, the goal recommended to the care target person is displayed in accordance with the past care track record of the care target person. Accordingly, it is possible to create a care plan that is effective in fulfilling the desire of the care target person.

The configuration 3 can also be similarly applied to a case where an issue or action item is recommended.

[Configuration 4] This configuration is a configuration that recommends a goal on the basis of a past care track record of another care target person. A care track record information table where a care track record of a care target person is registered, associated with an attribute of the care target person, is stored in the storage device 42. In the example of FIG. 12, the goal information table 420 further includes a field where a care track record or identification information thereof is registered. A care track record corresponding to an attribute having the same or a predetermined relationship with a care target person A is read from the care track record information table. One or more goals corresponding to a desire of the care target person A inputted in step S104 and to the care track record read out are read from the goal information table 420. The goals read out are displayed on the display device 44 as goals recommended to the care target person A. For example, if a goal Y is recommended as the next goal to a care target person B of an attribute having the same or predetermined relationship with the care target person A who carried out an action item X in the past, the action item X is registered as the care track record in the care track record information table. The goal Y of the goal information table 420 is registered, associated with the action item X.

Consequently, the goal recommended to the care target person A is displayed in accordance with the past care track record of the care target person B of the attribute having the same or predetermined relationship with the care target person A. Accordingly, it is possible to create a care plan that is effective in fulfilling the desire of the care target person A.

The configuration 4 can also be similarly applied to a case where an issue or action item is recommended.

[Configuration 5] This configuration is a configuration that recommends a goal on the basis of a track record of past care implemented by another care practitioner. A care track record information table where a track record of care implemented by a care practitioner for a care target person is registered, associated with an attribute of the care practitioner, is stored in the storage device 42. In the example of FIG. 12, the goal information table 420 further includes a field where a care track record or identification information thereof is registered. A care track record corresponding to an attribute having the same or a predetermined relationship with a care practitioner A is read from the care track record information table. One or more goals corresponding to a desire of a care target person for whom care is implemented by the care practitioner A, the desire being inputted in step S104, and to the care track record read out are read from the goal information table 420. The goals read out are displayed on the display device 44 as goals recommended to the care practitioner A. For example, if a goal Y is recommended as the next goal to a care practitioner B of an attribute having the same or predetermined relationship with the care practitioner A who carried out an action item X in the past, the action item X is registered as a care track record in the care track record information table. The goal Y of the goal information table 420 is registered, associated with the action item X.

Consequently, the goal recommended to the care target person for whom the care practitioner A implements care is displayed in accordance with the track record of the past care implemented by the care practitioner B of the attribute having the same or predetermined relationship with the care practitioner A. Accordingly, it is possible to create a care plan that is effective in fulfilling the desire of the care target person.

The configuration 5 can also be similarly applied to a case where an issue or action item is recommended.

[Configuration 6] This configuration is a configuration that recommends a goal in accordance with a difference between the achievement rate and the fulfillment rate. If the difference between the achievement rate inputted in step S124 and the fulfillment rate inputted in step S126, both of which are related to a care target person A, or a rank of the difference is equal to or less than, or equal to or greater than, a predetermined value, a goal recommended to the care target person A among one or more goals for fulfilling the desire of the care target person A inputted in step S104 is displayed on the display device 44 on the basis of the goal information table 420. For example, one or more goals for fulfilling a desire of a care target person are registered, associated with a difference between the achievement rate and the fulfillment rate, or a rank thereof, in the goal information table 420. The difference between the achievement rate inputted in step S124 and the fulfillment rate inputted in step S126, both of which are related to the care target person A, or the rank thereof is calculated. If the calculated difference or rank is equal to or less than, or equal to or greater than, the predetermined value, one or more goals corresponding to the calculated difference or rank are read from the goal information table 420. The goals read out are displayed on the display device 44 as goals recommended to the care target person A.

Consequently, if the difference between the achievement rate and the fulfillment rate, or the rank thereof is equal to less than, equal to or greater than, the predetermined value, a goal recommended to the care target person is displayed. Accordingly, it is possible to implement care in such a manner as to near the fulfillment of the desire of the care target person.

The configuration 6 can also be similarly applied to a case where an issue or action item is recommended.

[Configuration 7] This configuration is a configuration that recommends a goal if the difference between the achievement rate and the fulfillment rate is large in the configuration 6. One or more goals for fulfilling the desire of the care target person, and the degrees of difficulty in achieving the one or more goals are registered, associated with the desire, in the goal information table 420. In the example of FIG. 12, the goal information table 420 further includes a field where the degree of difficulty in achieving a goal is registered. If the difference between the achievement rate and the fulfillment rate is equal to or greater than a predetermined value, a goal having a different degree of difficulty in achievement from a previous input is displayed on the display device 44. For example, a goal having a lower degree of difficulty in achievement than the previous input is displayed.

Consequently, if the difference between the achievement rate and the fulfillment rate is equal to or greater than the predetermined value, a goal having a different degree of difficulty in achievement from the previous input is displayed. Accordingly, it is possible to implement care in such a manner as to near the fulfillment of the desire of the care target person.

The configuration 7 can also be similarly applied to a case where an issue or action item is recommended.

[Configuration 8A] This configuration is a configuration that determines whether or not the setting of a goal is appropriate from a past track record and inputted information. It is determined whether or not the goal inputted in step S110 can be achieved on the basis of care management information including a goal, an issue, and a target achievement date, which were inputted in the past, and the goal, the issue, and the target achievement date, which were inputted in steps S110, S114, and S120. For example, care management information including a goal, an issue, and a target achievement date, which were inputted in the past and include the same combination of a goal and an issue as the goal and the issue, which were inputted in steps S110 and S114, is read from the storage device 42. If the target achievement date inputted in step S120 is earlier than the target achievement date included in the care management information read out, it is determined that the goal inputted in step S110 cannot be achieved.

Consequently, it is determined whether or not the goal can be achieved, from the past track record and the inputted information. Accordingly, it is possible to prevent the creation of a care plan with a low possibility of fulfilling the desire of the care target person.

[Configuration 8B] This configuration is a configuration that determines whether or not the setting of a goal is appropriate, from a past track record and inputted information. It is determined whether or not the goal inputted in step S110 can be achieved on the basis of care management information including a goal, an action item, and a target achievement date, which were inputted in the past, and the goal, the action item, and the target achievement date, which were inputted in steps S110, S118, and S120. For example, care management information including a goal, an action item, and a target achievement date, which were inputted in the past and include the same combination of a goal and an action item as the goal and the action item, which were inputted in steps S110 and S118, is read from the storage device 42. If the target achievement date inputted in step S120 is earlier than the target achievement date included in the care management information read out, it is determined that the goal inputted in step S110 cannot be achieved.

Consequently, it is determined whether or not the goal can be achieved, from the past track record and the inputted information. Accordingly, it is possible to prevent the creation of a care plan with a low possibility of fulfilling the desire of the care target person.

[Configuration 9]

This configuration is a configuration that displays candidates for a target achievement date. Candidates for a target achievement date scheduled to achieve an inputted goal are displayed on the basis of a target achievement date information table where one or more target achievement dates scheduled to achieve a goal of the goal information table 420 are registered, associated with the goal. Any of the displayed target achievement dates is inputted. Care management information including the inputted target achievement date is registered in the storage device 42. The target achievement date is displayed per care target person on the basis of the care management information of the storage device 42.

Consequently, one or more target achievement dates scheduled to achieve the selected goal are displayed, and the care practitioner is simply required to select any of the target achievement dates. Accordingly, even a care practitioner with little experience can create a care plan for fulfilling the desire of the care target person compared to before.

[Configuration 10] This configuration is a configuration that displays candidates for an achievement rate. Candidates for an achievement rate of achievement of an inputted goal are displayed on the basis of an achievement rate information table where one or more achievement rates of a goal of the goal information table 420 are registered, associated with the goal. Any of the displayed achievement rates is inputted. Care management information including the inputted achievement rate is registered in the storage device 42. The achievement rate is displayed per care target person on the basis of the care management information of the storage device 42.

Consequently, one or more achievement rates of the selected goal are displayed, and the care practitioner is simply required to select any of the achievement rates. Accordingly, even a care practitioner with little experience can create a care plan for fulfilling the desire of the care target person compared to before.

[Configuration 11] This configuration is a configuration that displays candidates for a fulfillment rate. Candidates for a fulfillment rate obtained by achieving an inputted goal are displayed on the basis of a fulfillment rate information table where one or more fulfillment rates of a desire that is attempted to be fulfilled by achieving a goal of the goal information table 420 are registered, associated with the goal. Any of the displayed fulfillment rates is inputted. Care management information including the inputted fulfillment rate is registered in the storage device 42. The fulfillment rate is displayed per care target person on the basis of the care management information of the storage device 42.

Consequently, one or more fulfillment rates of the desire that is attempted to be fulfilled by achieving the selected goal is displayed, and the care practitioner is simply required to select any of the fulfillment rates. Accordingly, even a care practitioner with little experience can create a care plan for fulfilling the desire of the care target person compared to before.

[Configuration 12] This configuration is a configuration that creates a desire fulfillment history. For example, if the fulfillment rate reaches 100%, a desire fulfillment history including the date when the desire came true is created. Care management information including the created desire fulfillment history is registered in the storage device 42. Moreover, at this point in time, it can also be configured in such a manner as to prompt to set a new goal.

[Configuration 13] This configuration is a configuration that creates a goal achievement history. For example, if the achievement rate reaches 100%, a goal achievement history including the date when the goal was achieved is created. Care management information including the created goal achievement history is registered in the storage device 42. Moreover, at this point in time, it can also be configured in such a manner as to prompt to set a new goal.

Moreover, in terms of the configurations 2 to 7, it can also be configured in such a manner that a recommendation is made by AI. For example, the following configurations can be employed as the configuration using AI.

[Configuration 14] This configuration is a configuration that causes AI to present a goal. AI performs learning on the basis of, for example, learning data where a desire and a goal are associated. AI, which has performed learning, inputs a desire, and outputs one or more goals matching the inputted desire. Examples of the match include a goal appropriate to fulfill the inputted desire on the basis of the learning result (for example, a goal classified according to the inputted desire among a plurality of learned goals. In terms of the meaning of appropriate, the same shall apply below). The goals outputted by AI are presented.

[Configuration 15] This configuration is a configuration that causes AI to present an issue. AI performs learning on the basis of, for example, learning data where a desire, a goal, and an issue are associated. AI, which has performed learning, inputs at least a desire or a goal, and outputs one or more issues matching, for example, the inputted desire. Examples of the match include an issue appropriate to achieve a goal for fulfilling the inputted desire on the basis of the learning result. The issues outputted by AI are presented.

Moreover, AI performs learning on the basis of, for example, learning data where a desire and an issue are associated. AI, which has performed learning, inputs a desire, and outputs one or more issues matching the inputted desire. The issues outputted by AI are presented.

Moreover, AI performs learning on the basis of, for example, learning data where a goal and an issue are associated. AI, which has performed learning, inputs a goal, and outputs one or more issues matching the inputted goal. The issues outputted by AI are presented.

Moreover, AI performs learning on the basis of, for example, learning data where an issue and an action item are associated. AI, which has performed learning, inputs an action item, and outputs one or more issues matching the inputted action item. The issues outputted by AI are presented.

Moreover, AI performs learning on the basis of, for example, learning data where a desire, a goal, an issue, and an action item are associated. AI, which has performed learning, inputs at least a desire, a goal, or an action item, and outputs one or more issues matching, for example, the inputted desire. The issues outputted by AI are presented.

[Configuration 16] This configuration is a configuration that causes AI to present an action item. AI performs learning on the basis of, for example, learning data where a desire, a goal, and an action item are associated. AI, which has performed learning, inputs at least a desire or a goal, and outputs one or more action items matching, for example, the inputted desire. Examples of the match include an action item appropriate to achieve a goal for fulfilling the inputted desire on the basis of the learning result. The action items outputted by AI are presented.

Moreover, AI performs learning on the basis of, for example, learning data where a desire, an issue, and an action item are associated. AI, which has performed learning, inputs at least a desire or an issue, and outputs one or more action items matching, for example, the inputted desire. The action items outputted by AI are presented.

Moreover, AI performs learning on the basis of, for example, learning data where a goal, an issue, and an action item are associated. AI, which has performed learning, inputs at least a goal or an issue, and outputs one or more action items matching, for example, the inputted goal. The action items outputted by AI are presented.

Moreover, AI performs learning on the basis of, for example, learning data where a desire, a goal, an issue, and an action item are associated. AI, which has performed learning, inputs at least a desire, a goal, or an issue, and outputs one or more action items matching, for example, the inputted desire. The action items outputted by AI are presented.

[Configuration 17] This configuration is a configuration that causes AI to present a goal and an issue. AI performs learning on the basis of, for example, learning data where a desire, a goal, and an issue are associated. AI, which has performed learning, inputs a desire, and outputs one or more goals and one or more issues, which match the inputted desire. Examples of the match include a goal appropriate to fulfill the inputted desire and an issue appropriate to achieve the goal on the basis of the learning result. The goals and the issues, which have been outputted by AI, are presented.

Moreover, AI performs learning on the basis of, for example, learning data where a goal, an issue, and an action item are associated. AI, which has performed learning, inputs an action item, and outputs one or more goals and one or more issues, which match the inputted action item. The goals and the issues, which have been outputted by AI, are presented.

[Configuration 18] This configuration is a configuration that causes AI to present a goal and an action item. AI performs learning on the basis of, for example, learning data where a desire, a goal, and an action item are associated. AI, which has performed learning, inputs a desire, and outputs one or more goals and one or more action items, which match the inputted desire. Examples of the match include a goal appropriate to fulfill the inputted desire and an action item appropriate to achieve the goal on the basis of the learning result. The goals and the action items, which have been outputted by AI, are presented.

Moreover, AI performs learning on the basis of, for example, learning data where a goal, an issue, and an action item are associated. AI, which has performed learning, inputs an issue, and outputs one or more goals and one or more action items, which match the inputted issue. The goals and the action items, which have been outputted by AI, are presented.

Moreover, AI performs learning on the basis of, for example, learning data where a desire, a goal, an issue, and an action item are associated. AI, which has performed learning, inputs at least a desire or an issue, and outputs one or more goals and one or more action items, which match, for example, the inputted desire. The goals and the action items, which have been outputted by AI, are presented.

[Configuration 19] This configuration is a configuration that causes AI to present an issue and an action item. AI performs learning on the basis of, for example, learning data where a desire, an issue, and an action item are associated. AI, which has performed learning, inputs a desire, and outputs one or more issues and one or more action items, which match the inputted desire. Examples of the match include an issue appropriate to achieve a goal for fulfilling the inputted desire and an action item appropriate to achieve the goal for fulfilling the inputted desire on the basis of the learning result. The issues and the action items, which have been outputted by AI, are presented.

Moreover, AI performs learning on the basis of, for example, learning data where a goal, an issue, and an action item are associated. AI, which has performed learning, inputs a goal, and outputs one or more issues and one or more action items, which match the inputted goal. The issues and the action items, which have been outputted by AI, are presented.

Moreover, AI performs learning on the basis of, for example, learning data where a desire, a goal, an issue, and an action item are associated. AI, which has performed learning, inputs at least a desire or a goal, and outputs one or more issues and one or more action items, which match, for example, the inputted desire. The issues and the action items, which have been outputted by AI, are presented.

[Configuration 20] This configuration is a configuration that causes AI to present a goal, an issue, and an action item. AI performs learning on the basis of, for example, learning data where a desire, a goal, an issue, and an action item are associated. AI, which has performed learning, inputs a desire, and outputs one or more goals, one or more issues, and one or more action items, which match the inputted desire. Examples of the match include a goal appropriate to fulfill the inputted desire, an issue appropriate to achieve the goal, and an action item appropriate to achieve the goal on the basis of the leaning result. The goals, the issues, and the action items, which have been outputted by AI, are presented.

[Configuration 21] This configuration is a configuration that causes AI to present a desire, an issue, and an action item. AI performs learning on the basis of, for example, learning data where a desire, a goal, an issue, and an action item are associated. AI, which has performed learning, inputs a goal, and outputs one or more desires, one or more issues, and one or more action items, which match the inputted goal. Examples of the match include a desire appropriate to be attempted to be fulfilled by the inputted goal, an issue appropriate to achieve the inputted goal, and an action item appropriate to achieve the inputted goal on the basis of the learning result. The desires, the issues, and the action items, which have been outputted by AI, are presented.

[Configuration 22] This configuration is a configuration that causes AI to present a desire, a goal, and an issue. AI performs learning on the basis of, for example, learning data where a desire, a goal, an issue, and an action item are associated. AI, which has performed learning, inputs an action item, and outputs one or more desires, one or more goals, and one or more issues, which match the inputted action item. Examples of the match include a desire appropriate to be attempted to be fulfilled by a goal attempted to be achieved by the inputted action item, a goal appropriate to be attempted to be achieved by the inputted action item, and an issue attempted to be solved by the inputted action item on the basis of the learning result. The desires, the goals, and the issues, which have been outputted by AI, are presented.

[Configuration 23] This configuration is a configuration that causes AI to present a desire, a goal, and an action item. AI performs learning on the basis of, for example, learning data where a desire, a goal, an issue, and an action item are associated. AI, which has performed learning, inputs an issue, and outputs one or more desires, one or more goals, and one or more action items, which match the inputted issue. Examples of the match include a desire appropriate to be attempted to be fulfilled by a goal attempted to be achieved by solving the inputted issue, a goal appropriate to be attempted to be achieved by solving the inputted issue, and an action item appropriate to solve the inputted issue on the basis of the learning result. The desires, the goals, and the action items, which have been outputted by AI, are presented.

Moreover, in the above second and third embodiments and the modifications thereof, it is configured in such a manner that goal information indicating one or more goals for fulfilling a desire of a care target person is registered, associated with the desire, in the goal information table 420, and that candidates for a goal for fulfilling the desire of the care target person are displayed on the basis of the goal information table 420. However, the configuration is not limited to this. It can also be configured in such a manner that goal information indicating one or more goals for fulfilling a desire of a family of a care target person is registered, associated with the desire, in the goal information table 420, and that candidates for a goal for fulfilling the desire of the family of the care target person are displayed on the basis of the goal information table 420.

Moreover, in the above second and third embodiments and the modifications thereof, it is configured in such a manner that an action item is registered, associated with an issue, in the action item information table 460. However, the configuration is not limited to this. It can also be configured in such a manner that an action item is registered, associated with a goal. In a sense that an action item is necessary to achieve a goal, an action item is associated indirectly with a goal via an issue in the above second and third embodiments while an action item is associated directly with a goal in this modification.

Moreover, in the above second and third embodiments and the modifications thereof, it is configured in such a manner as to register information on four items: desire, goal, issue, and action item. However, the configuration is not limited to this. It can also be configured in such a manner as to register information on at least any of the items.

Moreover, in the above second embodiment and the modifications thereof, it is configured in such a manner as to selectively input information on four items: desire, goal, issue, and action item. However, the configuration is not limited to this. It can also be configured in such a manner as to selectively input information on at least any of the items. For example, the following configuration can be employed.

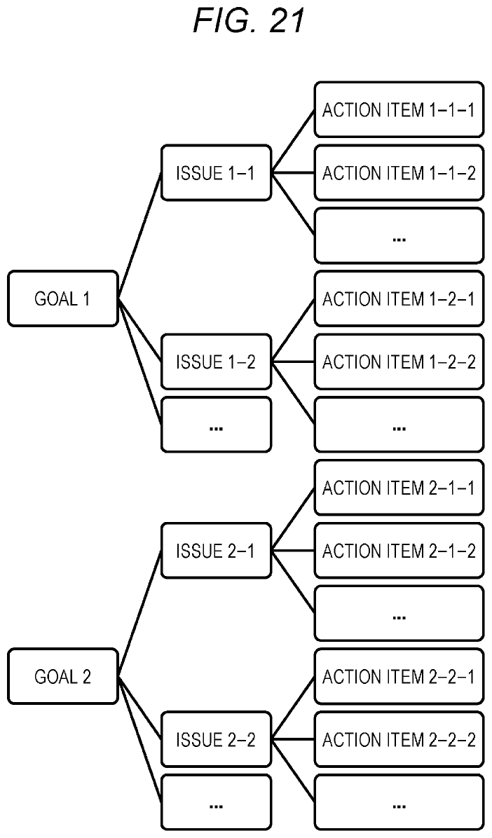

FIG. 21 is a conceptual diagram illustrating data coupling relationships of the tables 420, 440, and 460.

The example of FIG. 21 is a configuration that selectively inputs a goal, an issue, and an action item.

Care practitioners, mainly care practitioners with rich experience, list in advance (1) candidates for a goal for fulfilling a desire of a care target person, (2) candidates for an issue in achieving the goal, and (3) candidates for an action item necessary to achieve the goal.

As illustrated in FIG. 21, for one goal, one or more issues in achieving the goal can be set. In the example of FIG. 21, an issue 1-1, an issue 1-2, and other issues are set for a goal 1. An issue 2-1, an issue 2-2, and other issues are set for a goal 2. Each goal is associated with issues to be set. Accordingly, the tendency and the content may be common or similar in the issues 1-1, 1-2, 2-1, and 2-2. However, the issues 1-1, 1-2, 2-1, and 2-2 are basically independent of each other.

Next, for one issue, one or more action items necessary to solve the issue can be set. In the example of FIG. 21, an action item 1-1-1, an action item 1-1-2, and other action items are set for the issue 1-1. An action item 1-2-1, an action item 1-2-2, and other action items are set for the issue 1-2. An action item 2-1-1, an action item 2-1-2, and other action items are set for the issue 2-1. An action item 2-2-1, an action item 2-2-2, and other action items are set for the issue 2-2. Each issue is associated with action items to be set. Accordingly, the tendency and the content may be common or similar in the action items 1-1-1, 1-1-2, 1-2-1, 1-2-2, 2-1-1, 2-1-2, 2-2-1, and 2-2-2. However, the action items 1-1-1, 1-1-2, 1-2-1, 1-2-2, 2-1-1, 2-1-2, 2-2-1, and 2-2-2 are basically independent of each other.

This data structure is realized by the goal information table 420, the issue information table 440, and the action item information table 460. A goal is registered, associated with a goal ID, in the goal information table 420. Moreover, the desire information table 400 is not required.

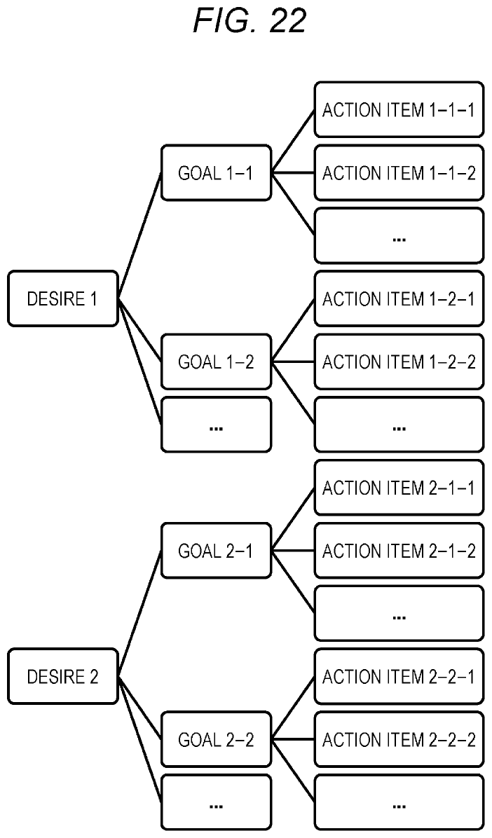

FIG. 22 is a conceptual diagram illustrating data coupling relationships of the tables 400, 420, and 460.

The example of FIG. 22 is a configuration that selectively inputs a desire, a goal, and an action item.

Care practitioners, mainly care practitioners with rich experience, list in advance (1) candidates for a desire of a care target person, (2) candidates for a goal for fulfilling the desire, and (3) candidates for an action item necessary to achieve the goal.

As illustrated in FIG. 22, for one desire, one or more goals for fulfilling the desire can be set. In the example of FIG. 22, a goal 1-1, a goal 1-2, and other goals are set for a desire 1. A goal 2-1, a goal 2-2, and other goals are set for a desire 2. Each desire is associated with goals to be set. Accordingly, the tendency and the content may be common or similar in the goals 1-1, 1-2, 2-1, and 2-2. However, the goals 1-1, 1-2, 2-1, and 2-2 are basically independent of each other.

Next, for one goal, one or more action items necessary to achieve the goal can be set. In the example of FIG. 22, an action item 1-1-1, an action item 1-1-2, and other action items are set for the goal 1-1. An action item 1-2-1, an action item 1-2-2, and other action items are set for the goal 1-2. An action item 2-1-1, an action item 2-1-2, and other action items are set for the goal 2-1. An action item 2-2-1, an action item 2-2-2, and other action items are set for the goal 2-2. Each goal is associated with action items to be set. Accordingly, the tendency and the content may be common or similar in the action items 1-1-1, 1-1-2, 1-2-1, 1-2-2, 2-1-1, 2-1-2, 2-2-1, and 2-2-2. However, the action items 1-1-1, 1-1-2, 1-2-1, 1-2-2, 2-1-1, 2-1-2, 2-2-1, and 2-2-2 are basically independent of each other.

This data structure is realized by the desire information table 400, the goal information table 420, and the action item information table 460. An action item is registered, associated with an action item ID and a goal ID, in the action item information table 460. Moreover, the issue information table 440 is not required.

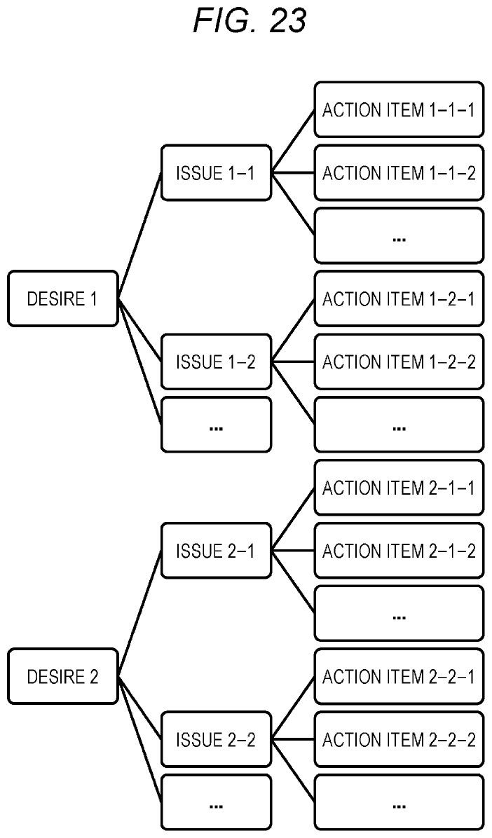

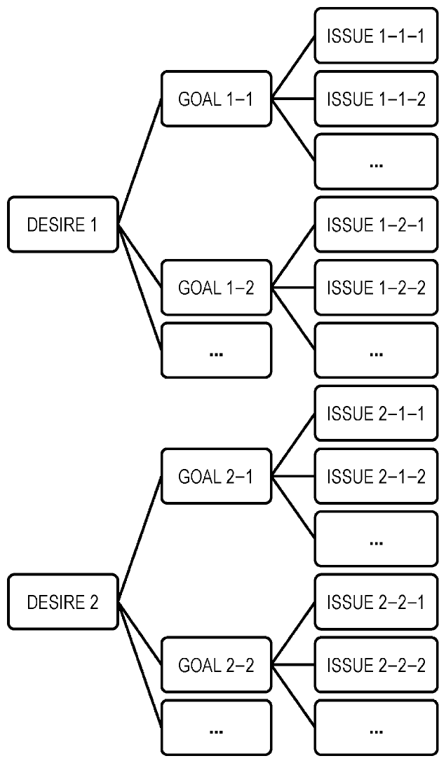

FIG. 23 is a conceptual diagram illustrating data coupling relationships of the tables 400, 440, and 460.

The example of FIG. 23 is a configuration that selectively inputs a desire, an issue, and an action item.

Care practitioners, mainly care practitioners with rich experience, list in advance (1) candidates for a desire of a care target person, (2) candidates for an issue in achieving a goal for fulfilling the desire, and (3) candidates for an action item necessary to solve the issue.

As illustrated in FIG. 23, for one desire, one or more issues in achieving a goal for fulfilling the desire can be set. In the example of FIG. 23, an issue 1-1, an issue 1-2, and other issues are set for a desire 1. An issue 2-1, an issue 2-2, and other issues are set for a desire 2. Each desire is associated with issues to be set. Accordingly, the tendency and the content may be common or similar in the issues 1-1, 1-2, 2-1, and 2-2. However, the issues 1-1, 1-2, 2-1, and 2-2 are basically independent of each other.

Next, for one issue, one or more action items necessary to solve the issue can be set. In the example of FIG. 23, an action item 1-1-1, an action item 1-1-2, and other action items are set for the issue 1-1. An action item 1-2-1, an action item 1-2-2, and other action items are set for the issue 1-2. An action item 2-1-1, an action item 2-1-2, and other action items are set for the issue 2-1. An action item 2-2-1, an action item 2-2-2, and other action items are set for the issue 2-2. Each issue is associated with action items to be set. Accordingly, the tendency and the content may be common or similar in the action items 1-1-1, 1-1-2, 1-2-1, 1-2-2, 2-1-1, 2-1-2, 2-2-1, and 2-2-2. However, the action items 1-1-1, 1-1-2, 1-2-1, 1-2-2, 2-1-1, 2-1-2, 2-2-1, and 2-2-2 are basically independent of each other.

This data structure is realized by the desire information table 400, the issue information table 440, and the action item information table 460. An issue is registered, associated with an issue ID and a desire ID, in the issue information table 440. Moreover, the goal information table 420 is not required.

FIG. 24 is a conceptual diagram illustrating data coupling relationships of the tables 400, 420, and 440.

The example of FIG. 24 is a configuration that selectively inputs a desire, a goal, and an issue.

Care practitioners, mainly care practitioners with rich experience, list in advance (1) candidates for a desire of a care target person, (2) candidates for a goal for fulfilling the desire, ad (3) candidates for an issue in achieving the goal.

As illustrated in FIG. 24, for one desire, one or more goals for fulfilling the desire can be set. In the example of FIG. 24, a goal 1-1, a goal 1-2, and other goals are set for a desire 1. A goal 2-1, a goal 2-2, and other goals are set for a desire 2. Each desire is associated with goals to be set. Accordingly, the tendency and the content may be common or similar in the goals 1-1, 1-2, 2-1, and 2-2. However, the goals 1-1, 1-2, 2-1, and 2-2 are basically independent of each other.

Next, for one goal, one or more issues in achieving the goal can be set. In the example of FIG. 24, an issue 1-1-1, an issue 1-1-2, and other issues are set for the goal 1-1. An issue 1-2-1, an issue 1-2-2, and other issues are set for the goal 1-2. An issue 2-1-1, an issue 2-1-2, and other issues are set for the goal 2-1. An issue 2-2-1, an issue 2-2-2, and other issues are set for the goal 2-2. Each issue is associated with issues to be set. Accordingly, the tendency and the content may be common or similar in the issues 1-1-1, 1-1-2, 1-2-1, 1-2-2, 2-1-1, 2-1-2, 2-2-1, and 2-2-2. However, the issues 1-1-1, 1-1-2, 1-2-1, 1-2-2, 2-1-1, 2-1-2, 2-2-1, and 2-2-2 are basically independent of each other.

This data structure is realized by the desire information table 400, the goal information table 420, and the issue information table 440. The action item information table 460 is not required.

Moreover, in the above second and third embodiments and the modifications thereof, one or more goals are registered, associated with one desire, in the goal information table 420. The configuration is not limited to this. It is also possible to register one or more goals, associating the goals with a plurality of desires. In other words, the correspondence is simply required to be n to m (n and m are integers equal to or greater than one). The same shall apply to the correspondence between the goal and the issue, the correspondence between the issue and the action item, and the correspondence between the goal and the action item.

Moreover, in the above second and third embodiments and the modifications thereof, the terms “goal” and “issue” are used. However, terms to be used are not limited to a goal and an issue. Even if a “long-term objective” is used instead of a “goal”, and a “short-term objective” instead of an “issue”, they have the same meanings.

Moreover, in the above second and third embodiments and the modifications thereof, the term “action item” is used. However, a term to be used is not limited to an action item. Even if, for example, an “action plan”, a “service”, a “service menu”, a “service item”, or a “service content” is used instead of an action item, they have the same meaning.

Moreover, in the above third embodiment and the modifications thereof, the processes of steps S204, S210, S216, and S222 are provided to prevent double registration in terms of the same combinations of a desire, a goal, an issue, and an action item. However, the configuration is not limited to this. Such processes may not be provided, or such processes may be provided only to part of the items: desire, goal, issue, and action item.

Moreover, in the above second and third embodiments and the modifications thereof, the care support apparatus 100 being a single apparatus is realized. However, not limited to the care support apparatus 100, but also a network system can be realized. A care support server and a plurality of user terminals are connected to a network such as the Internet. The care support server is configured including functions equivalent to the care support apparatus 100. The point of receiving information from the user terminal instead of input through the input device 40, and the point of transmitting information to the user terminal instead of display on the display device 44 are different from the care support apparatus 100.

Moreover, in the above second and third embodiments and the modifications thereof, it is configured in such a manner as to use the storage device 42 included in the care support apparatus 100. However, the configuration is not limited to this. It can also be configured in such a manner as to use an external storage device such as a database server.

Moreover, in the above second and third embodiments and the modifications thereof, a description is given of the case where the program stored in advance in the ROM 32 is executed when the processes illustrated in the flowcharts of FIGS. 15 and 20 are executed. However, the configuration is not limited to this. A program presenting the procedures may be read into the RAM 34 from a storage medium where the program is stored, and executed.

The storage medium is a semiconductor storage medium such as RAM or ROM, a magnetic storage-type storage medium such as an FD or HD, an optical reading-type storage medium such as a CD, CDV, LD, or DVD, or a magnetic storage-type/optical reading-type storage medium such as an MO, and includes every storage medium regardless of a reading method such as electronic, magnetic, and optical reading as long as it is a computer-readable storage medium.

Moreover, the present invention is not limited to the above second and third embodiments and the modifications thereof, and can also be applied to other cases within the scope that does not depart from the gist of the present invention. For example, the present invention can be applied to cases of supporting a care practitioner in implementing care (for example, taking care, protection, caregiving, and nursing) for a care target person not limited to in the caregiving field but also in the medical (for example, rehab or treatment) or (for example, physical, intellectual, or mental) welfare field. In this case, if a database is constructed for items of the relevant field, it can be linked to a common format.

Moreover, the above first to third embodiments and the modifications thereof can also be applied to each other. In other words, the above second or third embodiment, or the modification thereof, can be applied to the above first embodiment and the modifications thereof in any combination. Moreover, the above first or third embodiment, or the modification thereof, can be applied to the above second embodiment and the modifications thereof in any combination. Moreover, the above first or second embodiment, or the modification thereof, can be applied to the above third embodiment and the modifications thereof in any combination.

### DESCRIPTION OF REFERENCE SIGNS

100 Care support apparatus
30 CPU
32 ROM
34 RAM
38 I/F
40 Input device
42 Storage device
44 Display device
400 Desire information table
420 Goal information table
440 Issue information table
460 Action item information table
402, 404, 422 to 426, 442 to 446, 462 to 466 Field

1. A care support system that supports a care practitioner in implementing care for a care target person, the care support system comprising:
a desire input means configured to input a desire of the care target person or his/her family;
a goal presenting means configured to present one or more goals for fulfilling the desire inputted by the desire input means on the basis of goal information, which indicates one or more goals for fulfilling the desire of the care target person or his/her family, of a goal information storage means where the goal information is stored, associated with the desire;
a goal input means configured to input any of the goals presented by the goal presenting means;
an issue presenting means configured to present one or more issues in achieving the goal inputted by the goal input means on the basis of issue information, which indicates one or more issues in achieving the goal related to the goal information, of an issue information storage means where the issue information is stored, associated with the goal;
an issue input means configured to input any of the issues presented by the issue presenting means;
an identification information input means configured to input identification information for identifying the care target person;
a care management information registration means configured to register, in a care management information storage means, care management information including the identification information inputted by the identification information input means, the desire inputted by the desire input means, the goal inputted by the goal input means, and the issue inputted by the issue input means; and
a care management information presenting means configured to present the identification information, the desire, the goal, and the issue per care target person on the basis of the care management information of the care management information storage means.

2. The care support system according to claim 1, further comprising:
an achievement degree input means configured to input the degree of achievement of the goal inputted by the goal input means; and
a fulfillment degree input means configured to input the degree of fulfillment of the desire inputted by the desire input means, wherein
the care management information registration means registers, in the care management information storage means, the care management information including the identification information, the desire inputted by the desire input means, the goal inputted by the goal input means, the issue inputted by the issue input means, the degree of achievement inputted by the achievement degree input means, and the degree of fulfillment inputted by the fulfillment degree input means, and
the care management information presenting means presents the identification information, the desire, the goal, the issue, the degree of achievement, and the degree of fulfillment per care target person on the basis of the care management information of the care management information storage means.

3. The care support system according to claim 2, further comprising:
a target achievement date input means configured to input a target achievement date scheduled to achieve the goal inputted by the goal input means;
an update input means configured to input an update of a previous input;
an actual fulfillment date input means configured to input a fulfillment date when the desire inputted by the desire input means is actually fulfilled; and
an actual achievement date input means configured to input an achievement date when the goal inputted by the goal input means is actually achieved, wherein
the care management information registration means registers, in the care management information storage means, the care management information including the identification information, the desire inputted by the desire input means, the goal inputted by the goal input means, the issue inputted by the issue input means, the degree of achievement inputted by the achievement degree input means, the degree of fulfillment inputted by the fulfillment degree input means, the target achievement date inputted by the target achievement date input means, the update inputted by the update input means, the actual fulfillment date inputted by the actual fulfillment date input means, and the actual achievement date inputted by the actual achievement date input means, and
the care management information presenting means presents the identification information, the desire, the goal, the issue, the degree of achievement, the degree of fulfillment, the target achievement date, the update, the actual fulfillment date, and the actual achievement date per care target person on the basis of the care management information of the care management information storage means.

4. The care support system according to claim 2, wherein
the goal information storage means stores goal information indicating one or more goals for fulfilling the desire of the care target person or his/her family, and the degrees of difficulty in achieving the one or more goals, associating the goal information with the desire, and
upon the degree of achievement inputted by the achievement degree input means or the degree of fulfillment inputted by the fulfillment degree input means, both of which are related to the care target person, being equal to or less than a predetermined degree, the goal presenting means presents a goal whose degree of difficulty in achievement corresponding to the goal is equal to or less than a predetermined degree among one or more goals for fulfilling the desire of the care target person inputted by the desire input means, on the basis of the goal information of the goal information storage means.

5. The care support system according to claim 2, further comprising an action item presenting means configured to present an action item in accordance with the degree of achievement inputted by the achievement degree input means, or the degree of fulfillment inputted by the fulfillment degree input means, on the basis of action item information, which indicates an action item necessary to achieve the goal, of an action item information storage means where the action item information is stored, associated with the degree of achievement or the degree of fulfillment.

6. The care support system according to claim 1, further comprising a recommended goal presenting means configured to present a goal recommended to the care target person among one or more goals for fulfilling the desire of the care target person inputted by the desire input means, on the basis of care track record information, which indicates a care track record of the care target person, of a care track record information storage means where the care track record information is stored, and the goal information of the goal information storage means.

7. The care support system according to claim 1, further comprising:
an attribute information acquisition means configured to acquire attribute information indicating an attribute of a first care target person; and
a recommended goal presenting means configured to acquire care track record information indicating a care track record of the care target person, the care track record information corresponding to an attribute having the same or a predetermined relationship with the first care target person, from a care track record information storage means where the care track record information is stored, associated with an attribute of the care target person, on the basis of the attribute information acquired by the attribute information acquisition means, and to present a goal recommended to the first care target person among one or more goals for fulfilling a desire of the first care target person inputted by the desire input means, on the basis of the acquired care track record information and the goal information of the goal information storage means.

8. The care support system according to claim 1, further comprising:
an attribute information acquisition means configured to acquire attribute information indicating an attribute of a first care practitioner; and
a recommended goal presenting means configured to acquire care track record information indicating a track record of care implemented by the care practitioner for the care target person, the care track record information corresponding to an attribute having the same or a predetermined relationship with the first care practitioner, from a care track record information storage means where the care track record information is stored, associated with an attribute of the care practitioner, on the basis of the attribute information acquired by the attribute information acquisition means, and to present a goal recommended to the first care practitioner among one or more goals for fulfilling a desire of a care target person for whom care is implemented by the first care practitioner, the desire being inputted by the desire input means, on the basis of the acquired care track record information and the goal information of the goal information storage means.

9. The care support system according to claim 2, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or less than a predetermined value, present a goal recommended to the care target person among one or more goals for fulfilling the desire of the care target person inputted by the desire input means, on the basis of the goal information of the goal information storage means.

10. The care support system according to claim 2, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or greater than a predetermined value, present a goal recommended to the care target person among one or more goals for fulfilling the desire of the care target person inputted by the desire input means, on the basis of the goal information of the goal information storage means.

11. The care support system according to claim 10, wherein
the goal information storage means stores goal information indicating one or more goals for fulfilling the desire of the care target person or his/her family, and the degrees of difficulty in achieving the one or more goals, associating the goal information with the desire, and
upon the difference or the rank being equal to or greater than the predetermined value, the recommended goal presenting means presents a goal having a different degree of difficulty in achievement from a previous input by the goal input means on the basis of the goal information of the goal information storage means.

12. The care support system according to claim 3, further comprising a goal achievement determination means configured to acquire, from the care management information storage means, care management information including a goal, an issue, and a target achievement date, which were inputted in the past and include the same combination of a goal and an issue as the goal inputted by the goal input means and the issue inputted by the issue input means, and upon the target achievement date inputted by the target achievement date input means being earlier than a target achievement date included in the acquired care management information, determine that it fails to achieve the goal inputted by the goal input means.

13. (canceled)

14. A care support system that supports a care practitioner in implementing care for a care target person, the care support system comprising:
a desire input means configured to input a desire of the care target person or his/her family;
a goal presenting means configured to present one or more goals for fulfilling the desire inputted by the desire input means on the basis of goal information, which indicates one or more goals for fulfilling the desire of the care target person or his/her family, of a goal information storage means where the goal information is stored, associated with the desire;
a goal input means configured to input any of the goals presented by the goal presenting means;
an action item presenting means configured to present one or more action items necessary to achieve the goal inputted by the goal input means on the basis of action item information, which indicates one or more action items necessary to achieve the goal related to the goal information, of an action item information storage means where the action item information is stored, associated with the goal;
an action item input means configured to input any of the action items presented by the action item presenting means;
an identification information input means configured to input identification information for identifying the care target person;
a care management information registration means configured to register, in a care management information storage means, care management information including the identification information inputted by the identification information input means, the desire inputted by the desire input means, the goal inputted by the goal input means, and the action item inputted by the action item input means; and
a care management information presenting means configured to present the identification information, the desire, the goal, and the action item per care target person on the basis of the care management information of the care management information storage means.

15. The care support system according to claim 14, further comprising:
an issue presenting means configured to present one or more issues in achieving the goal inputted by the goal input means on the basis of issue information, which indicates one or more issues in achieving the goal related to the goal information, of an issue information storage means where the issue information is stored, associated with the goal; and
an issue input means configured to input any of the issues presented by the issue presenting means, wherein
the care management information registration means registers, in the care management information storage means, the care management information including the identification information inputted by the identification information input means, the desire inputted by the desire input means, the goal inputted by the goal input means, the issue inputted by the issue input means, and the action item inputted by the action item input means, and
the care management information presenting means presents the identification information, the desire, the goal, the issue, and the action item per care target person on the basis of the care management information of the care management information storage means.

16. The care support system according to claim 14, further comprising:
an achievement degree input means configured to input the degree of achievement of the goal inputted by the goal input means; and
a fulfillment degree input means configured to input the degree of fulfillment of the desire inputted by the desire input means, wherein
the care management information registration means registers, in the care management information storage means, the care management information including the identification information inputted by the identification information input means, the desire inputted by the desire input means, the goal inputted by the goal input means, the action item inputted by the action item input means, the degree of achievement inputted by the achievement degree input means, and the degree of fulfillment inputted by the fulfillment degree input means, and
the care management information presenting means presents the identification information, the desire, the goal, the action item, the degree of achievement, and the degree of fulfillment per care target person on the basis of the care management information of the care management information storage means.

17. The care support system according to claim 16, further comprising:
a target achievement date input means configured to input a target achievement date scheduled to achieve the goal inputted by the goal input means;
an update input means configured to input an update of a previous input;
an actual fulfillment date input means configured to input a fulfillment date when the desire inputted by the desire input means is actually fulfilled; and
an actual achievement date input means configured to input an achievement date when the goal inputted by the goal input means is actually achieved, wherein
the care management information registration means registers, in the care management information storage means, the care management information including the identification information inputted by the identification information input means, the desire inputted by the desire input means, the goal inputted by the goal input means, the action item inputted by the action item input means, the degree of achievement inputted by the achievement degree input means, the degree of fulfillment inputted by the fulfillment degree input means, the target achievement date inputted by the target achievement date input means, the update inputted by the update input means, the actual fulfillment date inputted by the actual fulfillment date input means, and the actual achievement date inputted by the actual achievement date input means, and
the care management information presenting means presents the identification information, the desire, the goal, the action item, the degree of achievement, the degree of fulfillment, the target achievement date, the update, the actual fulfillment date, and the actual achievement date per care target person on the basis of the care management information of the care management information storage means.

18. The care support system according to claim 16, wherein
the goal information storage means stores goal information indicating one or more goals for fulfilling the desire of the care target person or his/her family, and the degrees of difficulty in achieving the one or more goals, associating the goal information with the desire, and
upon the degree of achievement inputted by the achievement degree input means or the degree of fulfillment inputted by the fulfillment degree input means, both of which are related to the care target person, being equal to or less than a predetermined degree, the goal presenting means presents a goal whose degree of difficulty in achievement corresponding to the goal is equal to or less than a predetermined degree among one or more goals for fulfilling the desire of the care target person inputted by the desire input means, on the basis of the goal information of the goal information storage means.

19. The care support system according to claim 16, further comprising a second action item presenting means configured to present an action item in accordance with the degree of achievement inputted by the achievement degree input means, or the degree of fulfillment inputted by the fulfillment degree input means, on the basis of the action item information of the action item information storage means.

20. The care support system according to claim 14, further comprising a recommended goal presenting means configured to present a goal recommended to the care target person among one or more goals for fulfilling the desire of the care target person inputted by the desire input means on the basis of care track record information, which indicates a care track record of the care target person, of a care track record information storage means where the care track record information is stored, and the goal information of the goal information storage means.

21. The care support system according to claim 14, further comprising:
an attribute information acquisition means configured to acquire attribute information indicating an attribute of a first care target person; and
a recommended goal presenting means configured to acquire care track record information indicating a care track record of the care target person, the care track record information corresponding to an attribute having the same or a predetermined relationship with the first care target person, from a care track record information storage means where the care track record information is stored, associated with an attribute of the care target person, on the basis of the attribute information acquired by the attribute information acquisition means, and to present a goal recommended to the first care target person among one or more goals for fulfilling a desire of the first care target person inputted by the desire input means, on the basis of the acquired care track record information and the goal information of the goal information storage means.

22. The care support system according to claim 14, further comprising:
an attribute information acquisition means configured to acquire attribute information indicating an attribute of a first care practitioner; and
a recommended goal presenting means configured to acquire care track record information indicating a track record of care implemented by the care practitioner for the care target person, the care track record information corresponding to an attribute having the same or a predetermined relationship with the first care practitioner, from a care track record information storage means where the care track record information is stored, associated with an attribute of the care practitioner, on the basis of the attribute information acquired by the attribute information acquisition means, and to present a goal recommended to the first care practitioner among one or more goals for fulfilling a desire of a care target person for whom care is implemented by the first care practitioner, the desire being inputted by the desire input means, on the basis of the acquired care track record information and the goal information of the goal information storage means.

23. The care support system according to claim 16, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or less than a predetermined value, present a goal recommended to the care target person among one or more goals for fulfilling the desire of the care target person inputted by the desire input means, on the basis of the goal information of the goal information storage means.

24. The care support system according to claim 16, further comprising a recommended goal presenting means configured to, upon a difference between the degree of achievement inputted by the achievement degree input means and the degree of fulfillment inputted by the fulfillment degree input means, both of which are related to the care target person, or a rank of the difference being equal to or greater than a predetermined value, present a goal recommended to the care target person among one or more goals for fulfilling the desire of the care target person inputted by the desire input means, on the basis of the goal information of the goal information storage means.

25. The care support system according to claim 24, wherein
the goal information storage means stores goal information indicating one or more goals for fulfilling the desire of the care target person or his/her family, and the degrees of difficulty in achieving the one or more goals, associating the goal information with the desire, and
upon the difference or the rank being equal to or greater than the predetermined value, the recommended goal presenting means presents a goal having a different degree of difficulty in achievement from a previous input by the goal input means on the basis of the goal information of the goal information storage means.

26. The care support system according to claim 17, further comprising a goal achievement determination means configured to acquire, from the care management information storage means, care management information including a goal, an action item, and a target achievement date, which were inputted in the past and include the same combination of a goal and an action item as the goal inputted by the goal input means and the action item inputted by the action item input means, and upon the target achievement date inputted by the target achievement date input means being earlier than a target achievement date included in the acquired care management information, determine that it fails to achieve the goal inputted by the goal input means.

27. A care support system that supports a care practitioner in implementing care for a care target person, the care support system comprising:
a desire input means configured to input a desire of the care target person or his/her family;
an issue presenting means configured to present one or more issues in achieving a goal for fulfilling the desire inputted by the desire input means on the basis of issue information, which indicates one or more issues in achieving the goal for fulfilling the desire, of an issue information storage means where the issue information is stored, associated with the desire;
an issue input means configured to input any of the issues presented by the issue presenting means;
an action item presenting means configured to present one or more action items necessary to achieve the goal for fulfilling the desire inputted by the desire input means on the basis of action item information, which indicates one or more action items necessary to achieve the goal for fulfilling the desire, of an action item information storage means where the action item information is stored, associated with the desire;
an action item input means configured to input any of the action items presented by the action item presenting means;
an identification information input means configured to input identification information for identifying the care target person;
a care management information registration means configured to register, in a care management information storage means, care management information including the identification information inputted by the identification information input means, the desire inputted by the desire input means, the issue inputted by the issue input means, and the action item inputted by the action item input means; and
a care management information presenting means configured to present the identification information, the goal, the issue, and the action item per care target person on the basis of the care management information of the care management information storage means.

28. A care support system that supports a care practitioner in implementing care for a care target person, the care support system comprising:
a goal input means configured to input a goal for fulfilling a desire of the care target person or his/her family;
an issue presenting means configured to present one or more issues in achieving the goal inputted by the goal input means on the basis of issue information, which indicates one or more issues in achieving the goal, of an issue information storage means where the issue information is stored, associated with the goal;
an issue input means configured to input any of the issues presented by the issue presenting means;
an action item presenting means configured to present one or more action items necessary to achieve the goal inputted by the goal input means on the basis of action item information, which indicates one or more action items necessary to achieve the goal, of an action item information storage means where the action item information is stored, associated with the goal;
an action item input means configured to input any of the action items presented by the action item presenting means;
an identification information input means configured to input identification information for identifying the care target person;
a care management information registration means configured to register, in a care management information storage means, care management information including the identification information inputted by the identification information input means, the goal inputted by the goal input means, the issue inputted by the issue input means, and the action item inputted by the action item input means; and
a care management information presenting means configured to present the identification information, the goal, the issue, and the action item per care target person on the basis of the care management information of the care management information storage means.

29. A care support information registration system that registers information used by a care support system that supports a care practitioner in implementing care for a care target person, the care support information registration system comprising:
a care management information acquisition means configured to acquire care management information where a desire of the care target person or his/her family, a goal for fulfilling the desire, and an issue in achieving the goal are associated with each other;
a desire extraction means configured to extract the desire from the care management information acquired by the care management information acquisition means;
a goal extraction means configured to extract the goal from the care management information acquired by the care management information acquisition means;
an issue extraction means configured to extract the issue from the care management information acquired by the care management information acquisition means;
a goal information registration means configured to register goal information indicating the goal extracted by the goal extraction means in a goal information storage means, associating the goal information with the desire extracted by the desire extraction means; and
an issue information registration means configured to register issue information indicating the issue extracted by the issue extraction means in an issue information storage means, associating the issue information with the goal extracted by the goal extraction means.

30. The care support information registration system according to claim 29, wherein
in the care management information, the desire of the care target person or his/her family, the goal for fulfilling the desire, the issue in achieving the goal, and an action item necessary to achieve the goal are associated with each other, and
the care support information registration system further comprises:
an action item extraction means configured to extract the action item from the care management information acquired by the care management information acquisition means; and
an action item information registration means configured to register action item information indicating the action item extracted by the action item extraction means in an action item information storage means, associating the action item information with the goal extracted by the goal extraction means.

31. A care support information registration system that registers information used by a care support system that supports a care practitioner in implementing care for a care target person, the care support information registration system comprising:
a care management information acquisition means configured to acquire care management information where a desire of the care target person or his/her family, a goal for fulfilling the desire, and an action item necessary to achieve the goal are associated with each other;
a desire extraction means configured to extract the desire from the care management information acquired by the care management information acquisition means;
a goal extraction means configured to extract the goal from the care management information acquired by the care management information acquisition means;
an action item extraction means configured to extract the action item from the care management information acquired by the care management information acquisition means;
a goal information registration means configured to register goal information indicating the goal extracted by the goal extraction means in a goal information storage means, associating the goal information with the desire extracted by the desire extraction means; and
an action item information registration means configured to register action item information indicating the action item extracted by the action item extraction means in an action item information storage means, associating the action item information with the goal extracted by the goal extraction means.

32. A care support information registration system that registers information used by a care support system that supports a care practitioner in implementing care for a care target person, the care support information registration system comprising:
a care management information acquisition means configured to acquire care management information where a desire of the care target person or his/her family, an issue in achieving a goal for fulfilling the desire, and an action item necessary to achieve the goal for fulfilling the desire are associated with each other;
a desire extraction means configured to extract the desire from the care management information acquired by the care management information acquisition means;
an issue extraction means configured to extract the issue from the care management information acquired by the care management information acquisition means;
an action item extraction means configured to extract the action item from the care management information acquired by the care management information acquisition means;
an issue information registration means configured to register issue information indicating the issue extracted by the issue extraction means in an issue information storage means, associating the issue information with the desire extracted by the desire extraction means; and
an action item information registration means configured to register action item information indicating the action item extracted by the action item extraction means in an action item information storage means, associating the action item information with the desire extracted by the desire extraction means.

33. A care support information registration system that registers information used by a care support system that supports a care practitioner in implementing care for a care target person, the care support information registration system comprising:
a care management information acquisition means configured to acquire care management information where a goal for fulfilling a desire of the care target person or his/her family, an issue in achieving the goal, and an action item necessary to achieve the goal are associated with each other;
a goal extraction means configured to extract the goal from the care management information acquired by the care management information acquisition means;
an issue extraction means configured to extract the issue from the care management information acquired by the care management information acquisition means;
an action item extraction means configured to extract the action item from the care management information acquired by the care management information acquisition means;
an issue information registration means configured to register issue information indicating the issue extracted by the issue extraction means in an issue information storage means, associating the issue information with the goal extracted by the goal extraction means; and
an action item information registration means configured to register action item information indicating the action item extracted by the action item extraction means in an action item information storage means, associating the action item information with the goal extracted by the goal extraction means.

34.-40. (canceled)
